(19) United States
(12) Patent Application Publication
(10) Pub. No.: US 2016/0243145 A1
(43) Pub. Date: Aug. 25, 2016

(54) METHODS AND COMPOSITIONS FOR TREATING AND/OR PREVENTING MUCOSITIS

(71) Applicant: GLYCOMIMETICS, INC., Rockville, MD (US)

(72) Inventors: John L. MAGNANI, Gaithersburg, MD (US); John M. PETERSON, Slate Hill, NY (US); Ingrid G. WINKLER, Queensland (AU)

(73) Assignee: GLYCOMIMETICS, INC., Rockville, MD (US)

(21) Appl. No.: 15/025,730

(22) PCT Filed: Sep. 29, 2014
(86) PCT No.: PCT/US14/57978

§ 371 Date: Mar. 29, 2016

Related U.S. Application Data
(60) Provisional application No. 61/884,856, filed on Sep. 30, 2013.

(51) Int. Cl. A61K 31/7034; A61K 31/7048; A61K 45/06; A61K 31/7056

(52) U.S. Cl. CPC A61K 31/7034; A61K 31/7056; A61K 31/7048; A61K 45/06

(57) ABSTRACT

Methods for treating and/or preventing mucositis comprising administering to a subject in need thereof an effective amount of at least one compound chosen from E-selectin antagonists, pharmaceutically acceptable salts of E-selectin antagonists, prodrugs of E-selectin antagonists, and pharmaceutically acceptable salts of prodrugs of E-selectin antagonists, and compositions comprising at least one of such compound.

### CROSS REFERENCE TO RELATED APPLICATIONS

This application claims the benefit under 35 U.S.C. §119(e) to U.S. Provisional Application No. 61/884,856 filed Sep. 30, 2013, which application is incorporated by reference herein in its entirety.

### FIELD OF INVENTION

The present disclosure relates to methods for treating and/or preventing mucositis comprising administering to a subject in need thereof an effective amount of at least one compound chosen from E-selectin antagonists, pharmaceutically acceptable salts of E-selectin antagonists, prodrugs of E-selectin antagonists, and pharmaceutically acceptable salts of prodrugs of E-selectin antagonists, as well as to compositions comprising at least one such compound.

### BACKGROUND OF THE INVENTION

Mucositis is a serious and often very painful disorder involving inflammation and ulceration of the mucous membrane, such as those of the gastrointestinal tract, the oral and oropharyngeal cavities, as well as the bladder, ear, nasal, optical, vaginal, and rectal mucosa. It often arises as a complication of antineoplastic therapy, such as chemotherapy and/or radiation therapy. The goal of such therapies is to kill rapidly-dividing cancer cells; unfortunately, other cells may be killed by the treatment as well, including epithelial cells of the mucous membranes, which can lead to mucositis.

While the overall frequency of mucositis, as well as its severity, depends on factors including, for example, the chemotherapy regimen and on the treatment modality, it is believed that approximately half of all cancer patients undergoing therapy suffer some degree of mucositis. Mucositis is believed to occur, for example, in virtually all patients treated with radiation therapy for head and neck tumors, all patients receiving radiation along the GI tract, and approximately 40% of those subjected to radiation therapy and/or chemotherapy for tumors in other locations (e.g., leukemias or lymphomas). It is also is believed to be highly prevalent in patients treated with high dose chemotherapy and/or irradiation for the purpose of myeloablation, such as in preparation for stem cell or bone marrow transplantation.

Mucositis can adversely impact the quality of life of cancer patients. Patients may experience pain, erythema, and/or deep, diffuse ulcers than can cause difficulty speaking, eating, and swallowing. Patients may also experience nausea and/or gastro-enteritis. Severe mucositis can lead to the need for parenteral nutrition or hospitalization or to disruptions in cancer treatment, alterations in treatment dosages, and/or shifting to different modes of treatment.

Mucositis may also be accompanied by a severe risk of fever and infection, as it can lead to a breach in the otherwise protective linings of the oral mucosa and gastrointestinal tract. The alimentary canal and gastrointestinal tract are colonized by a vast array of microorganisms, and mucosal legions can provide a portal of entry for bacteria.

Current therapy for mucositis is largely palliative, including administration of antibiotics, antifungals, or anti-inflammatory agents combined with topical treatments containing compounds that modulate wound-healing and prevent infection. There is but a single medication approved for the treatment of mucositis, palifermin. It is approved for use, however, only in a limited subset of patients. (See Kepivance Prescribing Information, revised May 2013). Therefore, there is a need for additional therapeutics for treating and/or preventing mucositis.

### SUMMARY OF THE INVENTION

The present application discloses compounds chosen from E-selectin antagonists, pharmaceutically acceptable salts of E-selectin antagonists, prodrugs of E-selectin antagonists, and pharmaceutically acceptable salts of prodrugs of E-selectin antagonists, and pharmaceutical compositions comprising at least one such compound that may be useful for treating and/or preventing mucositis.

In some embodiments, the present disclosure is directed to methods for treating and/or preventing mucositis comprising administering to a subject in need thereof an effective amount of at least one compound chosen from E-selectin antagonists of Formula (I):

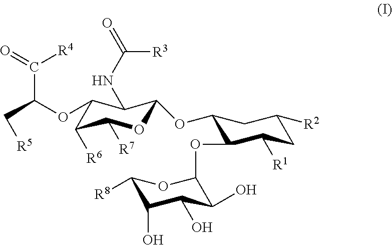

pharmaceutically acceptable salts of E-selectin antagonists of Formula (I), prodrugs of E-selectin antagonists of Formula (I), and pharmaceutically acceptable salts of prodrugs of E-selectin antagonists of Formula (I), wherein
R¹ is chosen from C₁₋₈ alkyl, C₂₋₈ alkenyl, C₂₋₈ alkynyl, C₁₋₈ haloalkyl, C₂₋₈ haloalkenyl and C₂₋₈ haloalkynyl groups;
R² is chosen from H, -M, and -L-M;
R³ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁴ is chosen from —OH and —NZ¹Z², wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl and C₂₋₈ haloalkynyl groups, wherein Z¹ and Z² may join together to form a ring;
R⁵ is chosen from C₃₋₈ cycloalkyl groups;
R⁶ is chosen from —OH, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁷ is chosen from —CH₂OH, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁸ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
L is chosen from linker groups; and
M is a non-glycomimetic moiety chosen from polyethylene glycol, thiazolyl, chromenyl, —C(═O)NH(CH₂)₁₋₄NH₂, C₁₋₈ alkyl, and —C(═O)OY, wherein Y is chosen from C₁₋₄ alkyl, C₂₋₄ alkenyl, and C₂₋₄ alkynyl groups.

As used herein, “compound of Formula (I)” includes an E-selectin antagonists of Formula (I), pharmaceutically acceptable salts of E-selectin antagonists of Formula (I), prodrugs of E-selectin antagonists of Formula (I), and pharmaceutically acceptable salts of prodrugs of E-selectin antagonists of Formula (I).

In some embodiments, the present disclosure is directed to methods for treating and/or preventing mucositis comprising administering to a subject in need thereof a pharmaceutical composition comprising an effective amount of at least one compound of Formula (I) and optionally at least one pharmaceutically acceptable ingredient.

In some embodiments, the present disclosure is directed to a use of at least one compound of Formula (I) in the manufacture of a medicament for treating and/or preventing mucositis.

### BRIEF DESCRIPTION OF THE DRAWINGS

FIG. 1 (FIG. 1A, FIG. 1B, FIG. 1C and FIG. 1D) is a diagram illustrating the synthesis of an embodiment (compound 25) of the at least one compound disclosed herein.

FIG. 2 is a diagram illustrating the synthesis of an embodiment of the at least one compound disclosed herein.

FIG. 3 illustrates the effect on small intestine weight (measure of inflammation) by an exemplary E-selectin antagonist, compound 25, after chemotherapy therapy.

FIG. 4 illustrates the effect on macrophage infiltration of the intestine by an exemplary E-selectin antagonist, compound 25, after radiation therapy.

### DETAILED DESCRIPTION

Disclosed herein are methods for treating and/or preventing (i.e., decreasing, inhibiting, and/or reducing the likelihood of occurrence in a statistical, biological, or clinically significant manner) mucositis, including mucositis in the gastrointestinal tract, the oral and oropharyngeal cavities, as well as the bladder, ear, nasal, optical, vaginal, and rectal mucosa, using at least one compound chosen from E-selectin antagonists, pharmaceutically acceptable salts of E-selectin antagonists, prodrugs of E-selectin antagonists, and pharmaceutically acceptable salts of prodrugs of E-selectin antagonists, or pharmaceutical compositions comprising the same.

In some embodiments, the present disclosure is directed to methods for treating and/or preventing mucositis comprising administering to a subject in need thereof at least one compound of Formula (I):

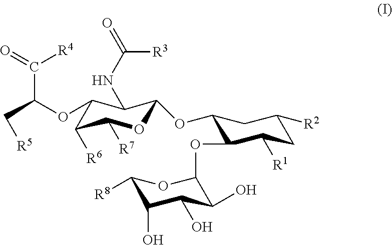

wherein each of R¹, R², R³, R⁴, R⁵, R⁶, R⁷ and R⁸ have the definitions described herein.

In some embodiments, the present disclosure is directed to methods for treating and/or preventing mucositis comprising administering to a subject in need thereof at least one compound of Formula (I),

wherein
R¹ is chosen from C₁₋₈ alkyl, C₂₋₈ alkenyl, alkynyl, C₁₋₈ haloalkyl, C₂₋₈ haloalkenyl and C₂₋₈ haloalkynyl groups;
R² is chosen from H, -M, and -L-M;
R³ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl;
R⁴ is chosen from —OH and —NZ¹Z², wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl and C₂₋₈ haloalkynyl groups, wherein Z¹ and Z² may join together to form a ring;
R⁵ is chosen from C₃₋₈ cycloalkyl groups;
R⁶ is chosen from —OH, C₁₋₈ alkyl, C₂₋₈ alkenyl, C₂₋₈ alkynyl, C₁₋₈ haloalkyl, C₂₋₈ haloalkenyl and C₂₋₈ haloalkynyl groups;
R⁷ is chosen from —CH₂OH, C₁₋₈ alkyl, C₂₋₈ alkenyl, C₂₋₈ alkynyl, C₁₋₈ haloalkyl, C₂₋₈ haloalkenyl and C₂₋₈ haloalkynyl groups;
R⁸ is chosen from C₁₋₈ alkyl, C₂₋₈ alkenyl, C₂₋₈ alkynyl, C₁₋₈ haloalkyl, C₂₋₈ haloalkenyl and C₂₋₈ haloalkynyl groups;
L is chosen from linker groups; and
M is a non-glycomimetic moiety chosen from polyethylene glycol, thiazolyl, chromenyl, —C(═O)NH(CH₂)₁₋₄NH₂, C₁₋₈ alkyl, and —C(═O)OY, wherein Y is chosen from C₁₋₄ alkyl, C₂₋₄ alkenyl and C₂₋₄ alkynyl groups.

In some embodiments, the at least one compound of Formula (I) is chosen from compounds wherein at least one of R¹, R³, R⁶, R⁷ and R⁸ is chosen from C₁₋₈ haloalkyl groups.

In some embodiments, the at least one compound of Formula (I) is chosen from compounds wherein at least one of R³, R⁶, R⁷ and R⁸ is chosen from C₁₋₈ haloalkyl groups.

In some embodiments, the at least one compound of Formula (I) is chosen from compounds wherein at least two of R¹, R³, R⁶, R⁷ and R⁸ are chosen from C₁₋₈ haloalkyl groups.

In some embodiments, the at least one compound of Formula (I) is chosen from compounds wherein R² is chosen from -L-M.

In some embodiments, the at least one compound of Formula (I) is chosen from compounds wherein at least one of R¹, R³, R⁶, R⁷ and R⁸ is chosen from C₁₋₈ haloalkyl groups, and R² is chosen from -L-M.

In some embodiments, the at least one compound of Formula (I) is chosen from compounds wherein each C₁₋₈ haloalkyl group is independently chosen from —CH₂X, —CH₂—(CH₂)ₘ—CH₂X, —CHX₂, —CH₂—(CH₂)ₘ—CHX₂, —CX₃ and —CH₂—(CH₂)ₘ—CX₃ groups, wherein each m is independently chosen from integers ranging from 1 to 6 and each X is independently chosen from F, Cl, Br and I. In some embodiments, the at least one compound of Formula (I) is chosen from compounds wherein at least one C₁₋₈ haloalkyl group is chosen from CH₂X, —CHX₂, and —CX₃ groups. In some embodiments, X is F.

In some embodiments, the at least one compound of Formula (I) is chosen from compounds wherein R¹ is chosen from C₁₋₈ alkyl, C₂₋₈ alkenyl, C₂₋₈ alkynyl, C₁₋₈ haloalkyl, C₂₋₈ haloalkenyl and C₂₋₃ haloalkynyl groups. In some embodiments, R¹ is chosen from C₁₋₈ alkyl and C₁₋₈ haloalkyl groups. In some embodiments, R¹ is chosen from C₁₋₃ alkyl and C₁₋₃ haloalkyl groups.

In some embodiments, the at least one compound of Formula (I) is chosen from compounds wherein R¹ is chosen from methyl (—CH₃), ethyl (CH₂CH₃), —CF₃ and —CHF₂. In some embodiments, R¹ is chosen from methyl (—CH₃) and —CHF₂.

In some embodiments, the at least one compound of Formula (I) is chosen from compounds wherein R² is chosen from H, -M, and -L-M, wherein M is chosen from C₁₋₈ alkyl, —C(═O)NH(CH₂)₁₋₄NH₂, polyethylene glycol (PEG), thiazolyl, chromenyl and —C(═O)OY, wherein Y is chosen from C₁₋₄ alkyl, C₂₋₄ alkenyl and C₂₋₄ alkynyl groups.

In some embodiments, the at least one compound of Formula (I) is chosen from compounds wherein R² is chosen from -M and -L-M, wherein M is polyethylene glycol. In some embodiments, R² is —C(═O)NH(CH₂)₂NH₂. In some embodiments, when R² is chosen from -M and -L-M, these moieties provide advantageous or improved characteristics such as enhanced bioavailability, desired pharmacokinetics, improved stability, and the like, to the compound and are non-immunogenic. Other exemplary non-glycomimetic moieties described herein include thiazolyl and chromenyl heteroaryls, for example 4-methylthiazolyl and 7-hydroxy-2H-chromen-2-on-yl. In some embodiments, R² is H.

R² may be attached to the glycomimetic portion of the compound of Formula (I) either directly or via a linker group. Linker groups (L) are well known to a person of ordinary skill in the art. In some embodiments, L is chosen from —C(═O)NH(CH₂)₁₋₄NHC(═O)—. In some embodiments, L is chosen from —C(═O)NH(CH₂)NHC(═O)— and —C(═O)NH(CH₂)₂NHC(═O)—. In some embodiments, L is chosen from —C(═O)NH(CH₂)₁₋₄NHC(═O)(CH₂)₁₋₄. In some embodiments, L is chosen from —C(═O)NH(CH₂)NHC(═O)—CH₂, and —C(═O)NH(CH₂)₂NHC(═O)—(CH₂)₂. Linker groups also include those called in the art “click chemistry” linkers (see, e.g., Brik et al., Chem. Bio. Chem. 2003, 4, 1246; Helms et al., J. Am. Chem. Soc. 2004, 126, 15020; Lober et al., Org. Lett. 2003, 5, 1753; Moses et al., Chem. Soc. Rev 2007, 36, 1249-1262) Other non-limiting examples of L are described in International Application Publication WO 2007/02850.

In some embodiments, the linker group is chosen from

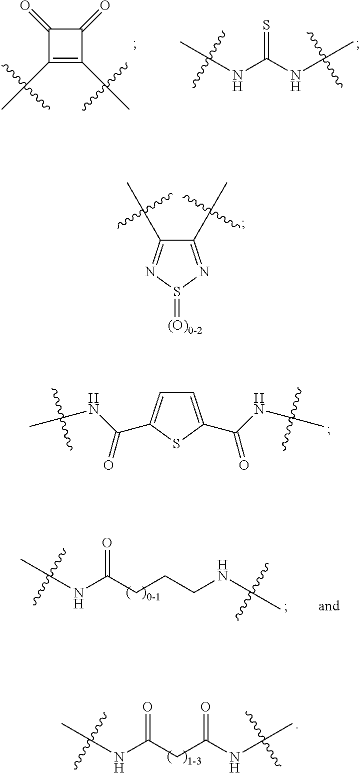

In some embodiments, the linker group is

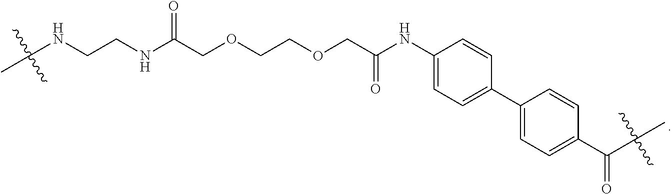 [SMILES: *NCCNC(=O)COCCOCC(=O)Nc1ccc(-c2ccc(C(*)=O)cc2)cc1]

In some embodiments, the linker group is

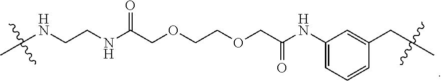 [SMILES: *Cc1cccc(NC(=O)COCCOCC(=O)NCCN*)c1]

In some embodiments, the linker group is chosen from —C(═O)—NH—(CH₂)₂—NH—, —CH₂—NH—CH₂—, and —C(═O)—NH—CH₂—.

In some embodiments, the at least one compound of Formula (I) is chosen from compounds wherein R³ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups. In some embodiments, R³ is chosen from C₁₋₈ alkyl, and C₁₋₈ haloalkyl groups. In some embodiments, R³ is chosen from C₁₋₃ alkyl and C₁₋₃ haloalkyl groups. In some embodiments, R³ is chosen from —CH₃ (methyl), —CH₂—CH₃ (ethyl), —CF₃ and —CHF₂. In some embodiments, R³ is chosen from methyl and trifluoromethyl.

In some embodiments, the at least one compound of Formula (I) is chosen from compounds wherein R⁴ is chosen from —OH and —NZ¹Z², wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl and C₇₋₈ haloalkynyl groups, wherein Z¹ and Z² may join together to form a ring. When Z¹ and Z² join together to form a ring, the ring is a heterocyclic ring wherein one or more heteroatoms is N. In some embodiments, R⁴ is chosen from —OH and —NZ¹Z², wherein Z¹ and Z², which may be identical or different, are independently chosen from H and C₁₋₈ alkyl groups. In some embodiments, —NZ¹Z² is —N(CH₃)₂.

In some embodiments, the at least one compound of Formula (I) is chosen from compounds wherein R⁵ is chosen from C₃₋₈ cycloalkyl groups (i.e., cyclopropyl, cyclobutyl, cyclopentyl, cyclohexyl, cycloheptyl or cyclooctyl). In some embodiments, R⁵ is chosen from C₃₋₆ cycloalkyl groups (i.e., cyclopropyl, cyclobutyl, cyclopentyl or cyclohexyl). In some embodiments, R⁵ is cyclohexyl.

In some embodiments, the at least one compound of Formula (I) is chosen from compounds wherein R⁶ is chosen from —OH, C₁₋₈ alkyl, C₂₋₈ alkenyl, C₂₋₈ alkynyl, C₁₋₈ haloalkyl, C₂₋₈ haloalkenyl and C₂₋₈ haloalkynyl groups. In some embodiments, R⁶ is —OH.

In some embodiments, at least one compound of Formula (I) is chosen from compounds wherein R⁷ is chosen from —CH₂OH, C₁₋₈ alkyl, C₂₋₈ alkenyl, C₂₋₈ alkynyl, C₁₋₈ haloalkyl, C₂₋₈ haloalkenyl and C₂₋₈ haloalkynyl groups. In some embodiments, R⁷ is chosen from —CH₂OH, C₁₋₈ alkyl, and C₁₋₈ haloalkyl groups. In some embodiments, R⁷ is chosen from —CH₂OH and —CH₃. In some embodiments, R⁷ is chosen from C₁₋₃ haloalkyl groups. In some embodiments, R⁷ is chosen from —CH₂F, —CHF₂ and —CF₃. In some embodiments, R⁷ is chosen from —CH₂OH and —CHF₂.

In some embodiments, the at least one compound of Formula (I) is chosen from compounds wherein R⁸ is chosen from C₁₋₈ alkyl, C₂₋₈ alkenyl, C₂₋₈ alkynyl, C₁₋₈ haloalkyl, C₂₋₈ haloalkenyl and C₂₋₈ haloalkynyl groups. In some embodiments, R⁸ is chosen from C₁₋₈ alkyl and C₁₋₈ haloalkyl groups. In some embodiments, R⁸ is chosen from C₁₋₃ alkyl and C₁₋₃ haloalkyl groups. In some embodiments, R⁸ is chosen from methyl (—CH₃), —CH₂F, —CHF₂, and trifluoromethyl (—CF₃). In some embodiments, R⁸ is chosen from methyl and trifluoromethyl (—CF₃).

In some embodiments, the at least one compound of Formula (I) is chosen from compounds wherein at least one or at least two of R¹, R³, R⁶, R⁷, and R⁸ is independently chosen from C₁₋₈ haloalkyl groups. In some embodiments, at least one of R³, R⁶, R⁷, and R⁸ is chosen from C₁₋₈ haloalkyl groups. In some embodiments, R² is chosen from -L-M. In some embodiments, R² is chosen from -L-M and at least one of R¹, R³, R⁶, R⁷ and R⁸ is chosen from C₁₋₈ haloalkyl groups. Oral bioavailability of a compound may be improved and/or the half-life of the compound increased when at least one of R¹, R³, R⁶, R⁷ and R⁸ is chosen from C₁₋₈ haloalkyl groups and R² is chosen from -M and -L-M.

In some embodiments, the methods for treating and/or preventing mucositis comprising administering to a subject in need thereof at least one compound of Formula (Ia):

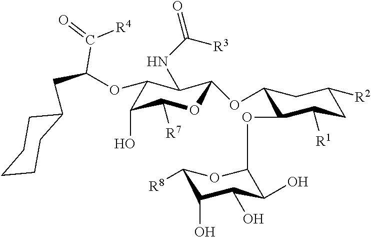

pharmaceutically acceptable salts of E-selectin antagonists of Formula (Ia), prodrugs of E-selectin antagonists of Formula (Ia), and pharmaceutically acceptable salts of prodrugs of E-selecting antagonists of Formula (Ia),
wherein
R¹ is chosen from C₁₋₈ alkyl and C₁₋₈ haloalkyl groups;
R² is chosen from H, -M, and -L-M;
R³ is chosen from C₁₋₈ alkyl, and C₁₋₈ haloalkyl groups;
R⁴ is chosen from —OH and —NZ¹Z² groups, wherein Z¹ and Z², which may be identical or different, are independently chosen from H and C₁₋₈ alkyl groups;
R⁷ is chosen from —CH₂OH, C₁₋₈ alkyl, and C₁₋₈ haloalkyl groups;
R⁸ is chosen from C₁₋₈ alkyl and C₁₋₈ haloalkyl groups;
L is chosen from linker groups; and
M is a non-glycomimetic moiety chosen from polyethylene glycol, thiazolyl, chromenyl, C₁₋₈ alkyl, —C(═O)NH(CH₂)₁₋₄NH₂ and —C(═O)OY, wherein Y is chosen from C₁₋₄ alkyl groups.

As used herein, “compound of Formula (Ia)” includes an E-selectin antagonists of Formula (Ia), pharmaceutically acceptable salts of E-selectin antagonists of Formula (Ia), prodrugs of E-selectin antagonists of Formula (Ia), and pharmaceutically acceptable salts of prodrugs of E-selectin antagonists of Formula (Ia).

In some embodiments, the at least one compound of Formula (Ia) is chosen from compounds wherein the haloalkyl group is a fluoroalkyl group. In some embodiments, R¹ is chosen from —CH₃, —CH₂CH₃, —CH₂F, —CHF₂, —CF₃, —CH₂CH₂F, —CH₂CHF₂, and —CH₂CF₃. In some embodiments, R³ is chosen from —CH₃, —CH₂F, —CHF₂, and —CF₃. In some embodiments, R⁴ is chosen from —OH and —N(CH₃)₂. In some embodiments, R⁷ is chosen from —CH₂OH, —CH₃, —CH₂F, —CHF₂, and —CF₃. In some embodiments, R⁸ is chosen from —CH₃, —CH₂F, —CHF₂, and —CF₃.

In some embodiments, the present disclosure is directed to methods for treating and/or preventing mucositis comprising administering to a subject in need thereof an effective amount of at least one compound of Formula (I),

wherein
R¹ is chosen from ethyl, CF₃, and —CHF₂;
R² is chosen from H, -M, and -L-M;
R³ is chosen from methyl and —CF₃;
R⁴ is chosen from —OH and —N(CH₃)₂;
R⁵ is cyclohexyl;
R⁶ is —OH;
R⁷ is chosen from —CH₂—OH, —CHF₂, and CF₃;
R⁸ is chosen from methyl, —CF₃, and —CHF₂;
L is chosen from linker groups; and
M is a non-glycomimetic moiety chosen from polyethylene glycol, thiazolyl, chromenyl, —C(═O)NH(CH₂)₁₋₄NH₂, C₁₋₈ alkyl, and —C(═O)OY, wherein Y is chosen from C₁₋₄ alkyl, C₂₋₄ alkenyl and C₂₋₄ alkynyl groups.

In some embodiments, the present disclosure is directed to methods for treating and/or preventing mucositis comprising administering to a subject in need thereof an effective amount of at least one compound of Formula (I) chosen from

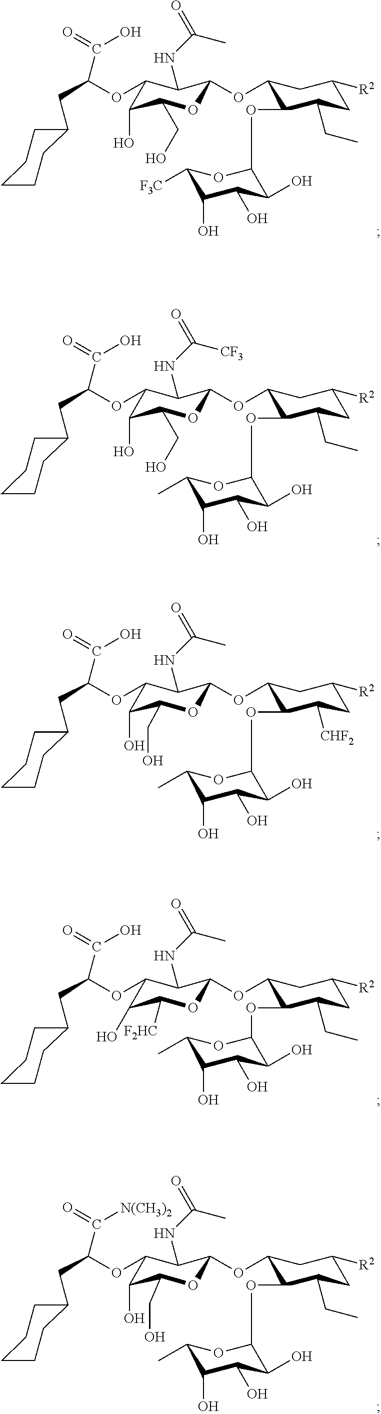

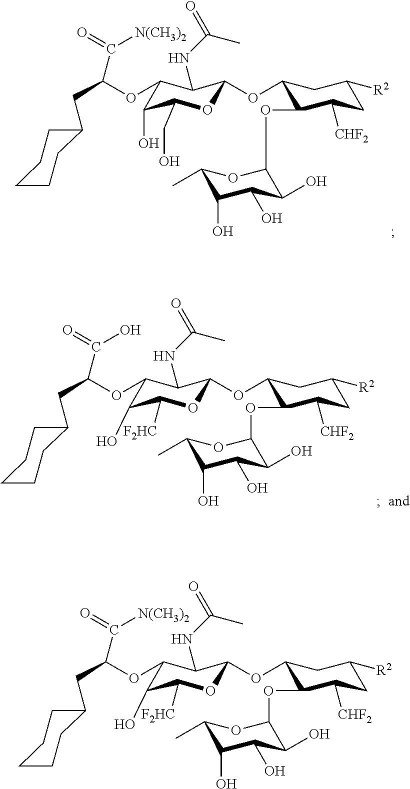

and pharmaceutically acceptable salts thereof, prodrugs thereof, and pharmaceutically acceptable salts of prodrugs thereof.

In some embodiments, the at least one compound of Formula (Ia) is chosen from compounds wherein R² is chosen from H, —C(═O)NH(CH₂)₂NH₂, and —C(═O)OCH₃.

In some embodiments, the at least one compound of Formula (I) is chosen from

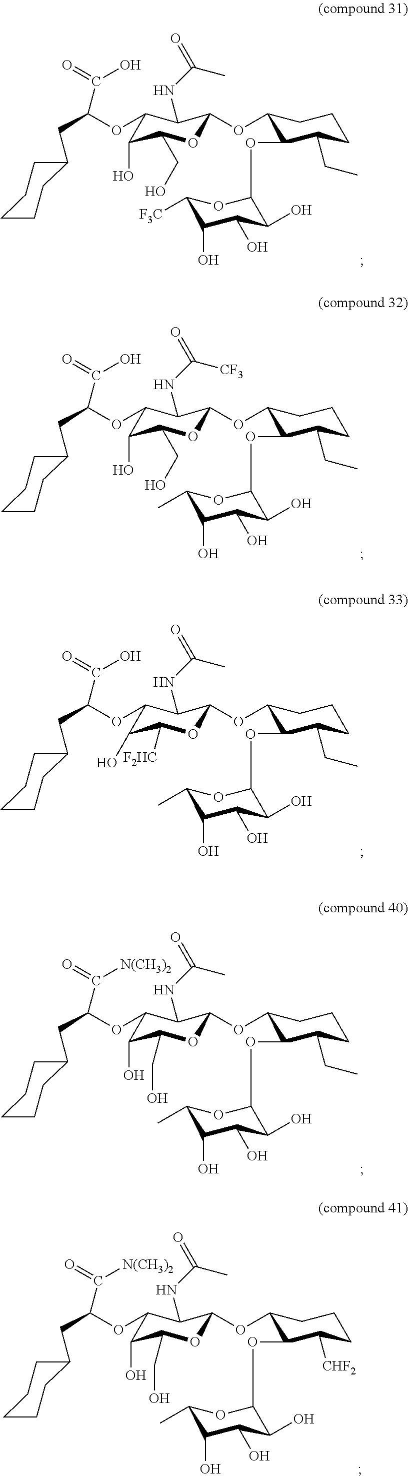

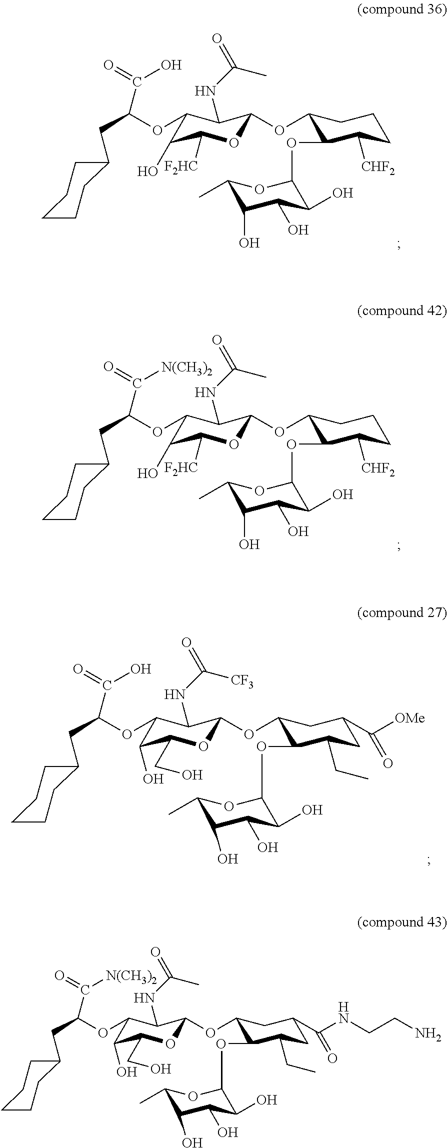

and pharmaceutically acceptable salts thereof, prodrugs thereof, and pharmaceutically acceptable salts of prodrugs thereof.

In some embodiments, the at least one compound of Formula (I) is chosen from

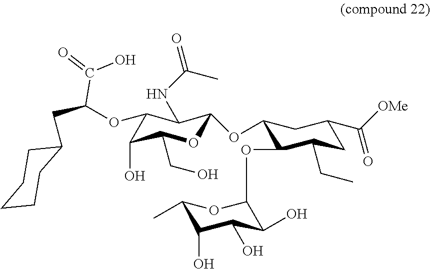

and pharmaceutically acceptable salts thereof, prodrugs thereof, and pharmaceutically acceptable salts of prodrugs thereof.

In some embodiments, the at least one compounds of Formula (I) and at least one compound of Formula (Ia) is chosen from compounds wherein R² is -M, wherein M is a polyethylene glycol (PEG). PEG is a polymer of repeating ethylene oxide units. Length and thus molecular weight vary depending upon how many of repeating units are present. The ethylene oxide units are abbreviated herein as

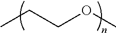

where n is chosen from integers ranging from 1 to 100. In some embodiments, n is chosen from 4, 8, 12, 16, 20, 24, and 28.

In some embodiments, the at least one compound of Formula (I) is chosen from compounds wherein R² is -L-M, wherein M is PEG and L is —C(═O)NH(CH₂)₂NHC(═O)— to provide one of the following compounds:

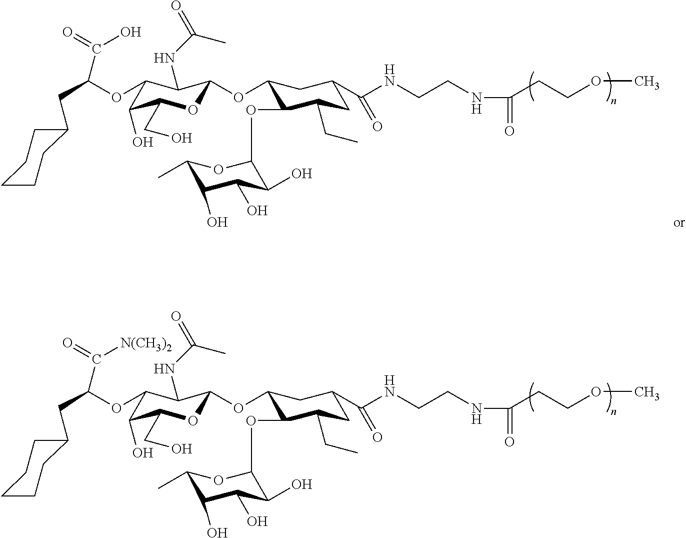

wherein n chosen from integers ranging from 1 to 100. In some embodiments, n is chosen from 4, 8, 12, 16, 20, 24, and 28.

In some embodiments, the at least one compound of Formula (I) is chosen from compounds wherein R² is -L-M, wherein M is PEG and L is —C(═O)NH(CH₂)₂NHC(═O)— to provide one of the following compounds:

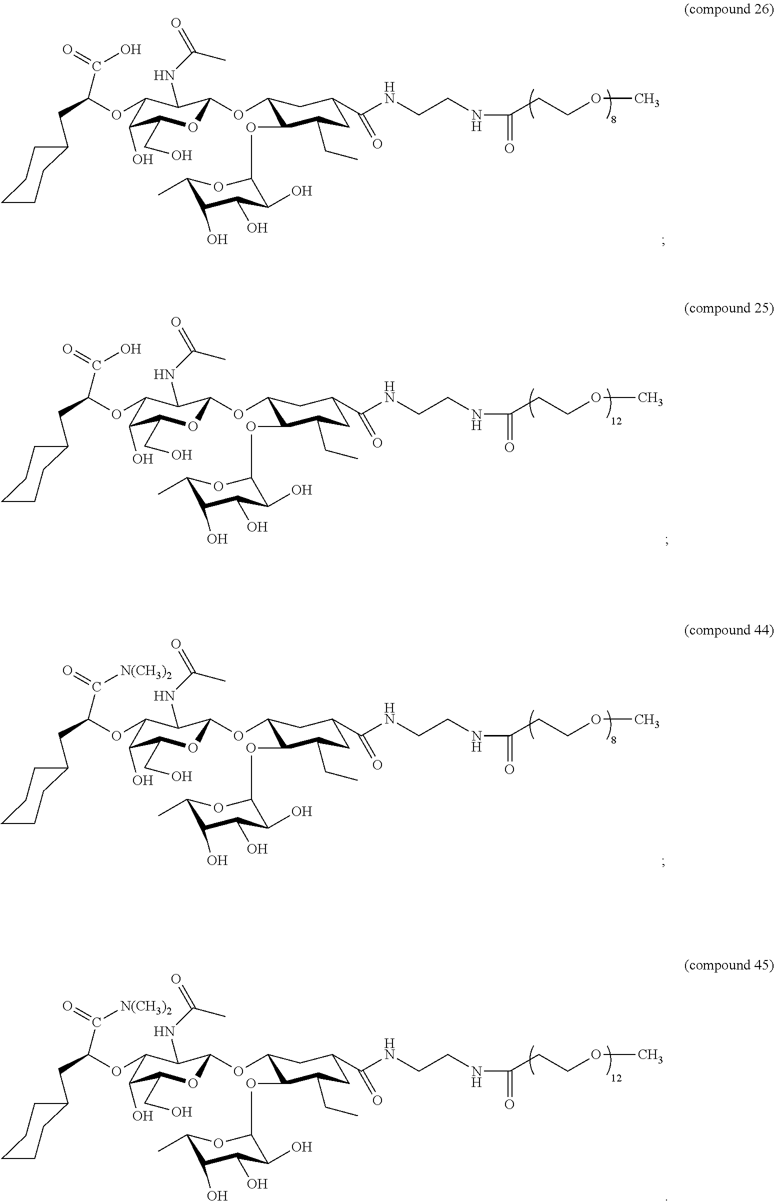

In some embodiments, the at least one compound of Formula (I) is chosen from compounds wherein R² is -L-M, wherein M is chosen from thiazolyl and chromenyl, for example, 4-methylthiazolyl or 7-hydroxy-2H-chromen-2-on-yl to provide one of the following compounds:

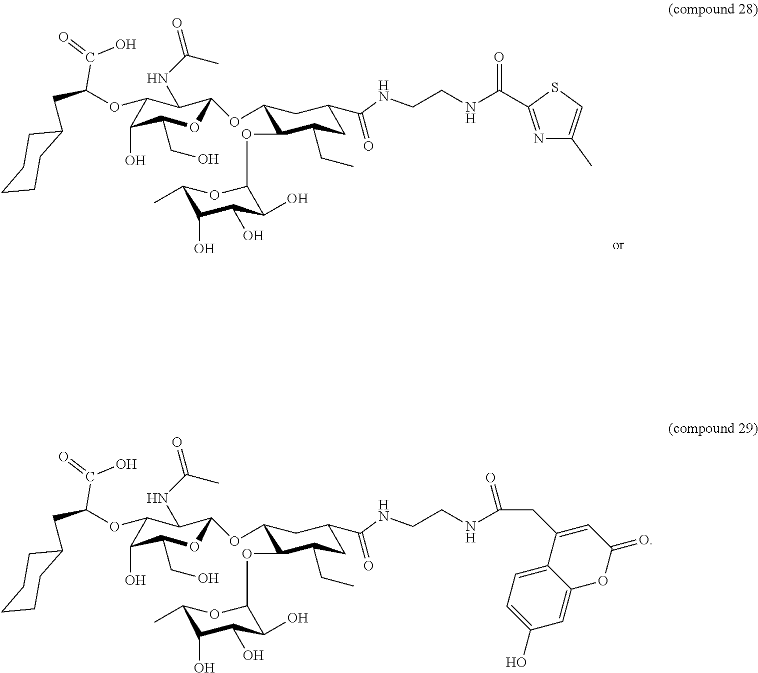

Also provided are pharmaceutical compositions comprising at least one compound of Formula (I). Such pharmaceutical compositions are described in greater detail herein. These compounds and compositions may be used in the methods described herein.

In some embodiments, at least one compound of Formula (I) may be used in the manufacture of a medicament for treating and/or preventing mucositis.

In some embodiments, at least one compound of Formula (I) or a pharmaceutical composition comprising at least one compound of Formula (I) may be used in methods described herein for decreasing the likelihood of occurrence of mucositis in a subject (i.e., individual, patient) who is in need thereof by administering the compound or composition to the subject.

In some embodiments, the compounds described herein and pharmaceutical compositions comprising at least one such compound may be used for treating and/or preventing mucositis.

In some embodiments, the compounds described herein and pharmaceutical compositions comprising at least one such compound may be used for reducing the number of days the patient is afflicted with mucositis.

In some embodiments, the mucositis is chosen from oral mucositis, esophageal mucositis, and gastrointestinal mucositis.

In some embodiments, the mucositis is alimentary mucositis.

In some embodiments, the subject is afflicted with cancer.

In some embodiments, the subject is afflicted with a cancer chosen from head and neck cancer, breast cancer, lung cancer, ovarian cancer, prostate cancer, lymphatic cancer, leukemic cancer, and/or gastrointestinal cancer.

In some embodiments, the mucositis is associated with radiation therapy and/or chemotherapy.

In some embodiments, the chemotherapy comprises administering a therapeutically effective amount of at least one compound chosen from platinum, cisplatin, carboplatin, oxaliplatin, mechlorethamine, cyclophosphamide, chlorambucil, azathioprine, mercaptopurine, vincristine, vinblastine, vinorelbine, vindesine, etoposide, teniposide, paclitaxel, docetaxel, irinotecan, topotecan, amsacrine, etoposide, etoposide phosphate, teniposide, 5-fluorouracil (5-FU), leucovorin, methotrexate, gemcitabine, taxane, leucovorin, mitomycin C, tegafur-uracil, idarubicin, fludarabine, mitoxantrone, ifosfamide and doxorubicin.

In some embodiments, the method further comprising a therapeutically effective amount of at least one MMP inhibitor, inflammatory cytokine inhibitor, mast cell inhibitor, NSAID, NO inhibitor, or antimicrobial compound.

In some embodiments, the method further comprising a therapeutically effective amount of velafermin and/or palifermin.

### DEFINITIONS

Whenever a term in the specification is identified as a range (e.g., C₁₋₄ alkyl), the range independently discloses and includes each element of the range. As a non-limiting example, C₁₋₄ alkyls includes, independently, C₁ alkyls, C₂ alkyls, C₃ alkyls, and C₄ alkyls.

The term “at least one” refers to one or more, such as one, two, etc. For example, the term “at least one C₁₋₄ alkyl” refers to one or more C₁₋₄ alkyl groups, such as one C₁₋₄ alkyl group, two C₁₋₄ alkyl groups, etc.

The term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted.

The term “alkenyl” includes straight, branched, and cyclic hydrocarbon groups comprising at least one double bond. The double bond of an alkenyl group can be unconjugated or conjugated with another unsaturated group. Non-limiting examples of alkenyl groups include vinyl, allyl, butenyl, pentenyl, hexenyl, butadienyl, pentadienyl, hexadienyl, 2-ethylhexenyl, and cyclopent-1-en-1-yl. Unless stated otherwise specifically in the specification, an alkenyl group may be optionally substituted.

The term “alkynyl” includes straight and branched hydrocarbon groups comprising at least one triple bonds. The triple bond of an alkynyl group can be unconjugated or conjugated with another unsaturated group. Non-limiting examples of alkynyl groups include ethynyl, propynyl, butynyl, pentynyl, and hexynyl. Unless stated otherwise specifically in the specification, an alkynyl group may be optionally substituted.

The term “aryl” includes hydrocarbon ring system group comprising, 6 to 30 carbon ring atoms and at least one aromatic ring. The aryl group may be a monocyclic, bicyclic, tricyclic or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The term “cycloalkyl” includes saturated monocyclic or polycyclic hydrocarbon group, which may include fused or bridged ring systems. Non-limiting examples of a cycloalkyl group include cyclopropyl, cyclobutyl, cyclopentyl, cyclohexyl, cycloheptyl, cyclooctyl, adamantyl, and norbornyl. Unless otherwise stated specifically in the specification, a cycloalkyl group may be optionally substituted.

The term “E-selectin antagonist” includes inhibitors of E-selectin only, as well as inhibitors of E-selectin and either P-selectin or L-selectin, and inhibitors of E-selectin, P-selectin, and L-selectin.

The term “fused” includes any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

The term “glycomimetic” includes any naturally occurring or non-naturally occurring carbohydrate compound in which at least one substituent has been replaced, or at least one ring has been modified (e.g., substitution of carbon for a ring oxygen), to yield a compound that is not fully carbohydrate.

The term “halo” or “halogen” includes fluoro, chloro, bromo and iodo.

The term “haloalkyl” includes alkyl groups, as defined herein, substituted by at least one halogen, as defined herein. Non-limiting examples include trifluoromethyl, difluoromethyl, trichloromethyl, 2,2,2-trifluoroethyl, 1,2-difluoroethyl, 3-bromo-2-fluoropropyl, and 1,2-dibromoethyl. A “fluoroalkyl” is a haloalkyl that is substituted with at least one fluoro group. Unless stated otherwise specifically in the specification, a haloalkyl group may be optionally substituted.

The term “haloalkenyl” includes alkenyl groups, as defined herein, substituted by at least one halogen, as defined herein. Non-limiting examples include fluoroethenyl, 1,2-difluoroethenyl, 3-bromo-2-fluoropropenyl, and 1,2-dibromoethenyl. A “fluoroalkenyl” is a haloalkenyl substituted with at least one fluoro group. Unless stated otherwise specifically in the specification, a haloalkenyl group may be optionally substituted.

The term “haloalkynyl” includes alkynyl groups, as defined herein, substituted by at least one halogen, as defined herein. Non-limiting examples include fluoroethynyl, 1,2-di fluoroethynyl, 3-bromo-2-fluoropropynyl, and 1,2-dibromoethynyl. A “fluoroalkynyl” is a haloalkynyl substituted with at least one fluoro group. Unless stated otherwise specifically in the specification, a haloalkynyl group may be optionally substituted.

The term “heterocyclyl” or “heterocyclic ring” includes 3- to 24-membered saturated or partially unsaturated non-aromatic ring groups comprising 2 to 23 ring carbon atoms and 1 to 8 ring heteroatom(s) each independently chosen from N, O, and S. Unless stated otherwise specifically in the specification, the heterocyclyl groups may be a monocyclic, bicyclic, tricyclic or tetracyclic ring system, which may include fused or bridged ring systems; and the nitrogen, carbon or sulfur atoms in the heterocyclyl group may be optionally oxidized; the nitrogen atom may be optionally quaternized; and the heterocyclyl group may be partially or fully saturated. Non-limiting examples include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl(benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

The term “non-glycomimetic moiety” includes moieties having a structure not intended to mimic a carbohydrate molecule. A non-glycomimetic moiety may not be (and is typically not) active as an E selectin antagonist. Instead, non-glycomimetic moieties are generally moieties added to a glycomimetic moiety for purposes of altering at least one property such as solubility, bio-availability, lipophilicity and/or other drug-like properties of the glycomimetic.

The term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

The term “steroid” or “steroidal moiety” includes compounds and moieties that contain a characteristic arrangement of four cycloalkane rings that are joined to each other. The core of a steroid comprises twenty carbon atoms bonded together that take the form of four fused rings: three cyclohexane rings and one cyclopentane ring. Non-limiting examples of a steroidal moiety include cholic acid, cholesterol and derivatives thereof.

The term “substituted” includes the situation where, in any of the above groups, at least one hydrogen atom is replaced by a non-hydrogen atom such as, for example, a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

The present disclosure includes within its scope all the possible geometric isomers, e.g. Z and E isomers (cis and trans isomers), of the compounds as well as all the possible optical isomers, e.g. diastereomers and enantiomers, of the compounds. Furthermore, the present disclosure includes in its scope both the individual isomers and any mixtures thereof, e.g. racemic mixtures. The individual isomers may be obtained using the corresponding isomeric forms of the starting material or they may be separated after the preparation of the end compound according to conventional separation methods. For the separation of optical isomers, e.g. enantiomers, from the mixture thereof conventional resolution methods, e.g. fractional crystallization, may be used.

The present disclosure includes within its scope all possible tautomers. Furthermore, the present disclosure includes in its scope both the individual tautomers and any mixtures thereof.

Some of the crystalline forms of any compound described herein may exist as polymorphs, which are also included and contemplated by the present disclosure. In addition, some of the compounds may form hydrates with water or solvates with other solvents. Such hydrates and solvates are similarly included within the scope of compounds and compositions described herein.

### Compound Synthesis Procedures

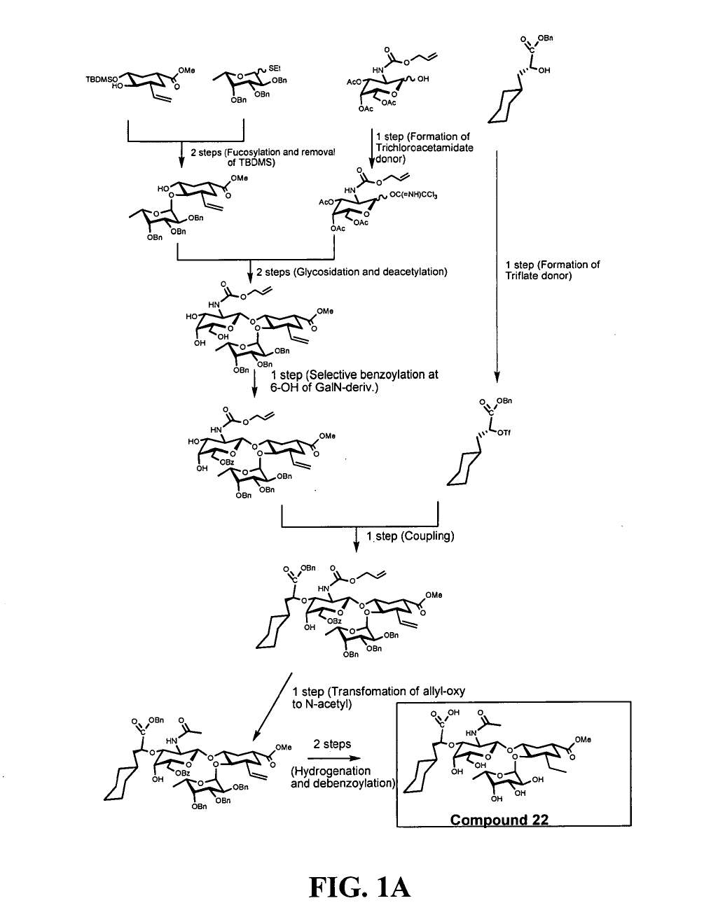

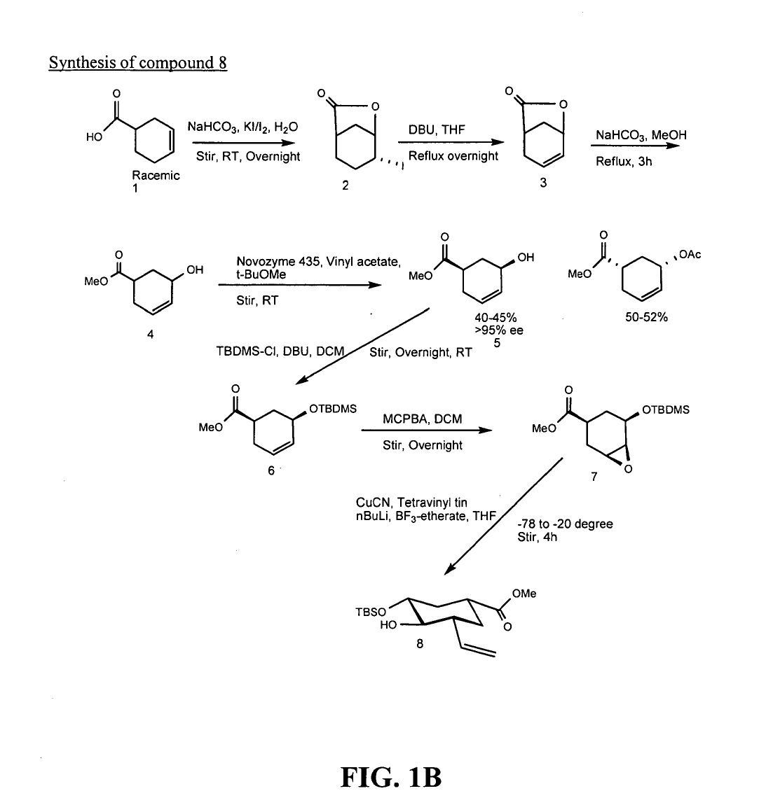

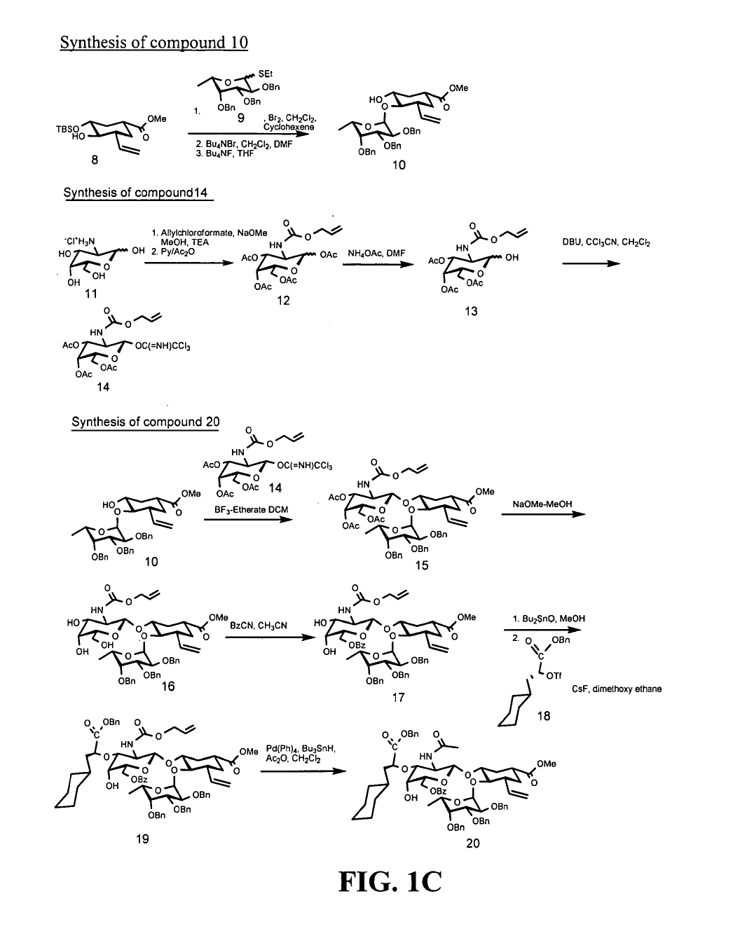

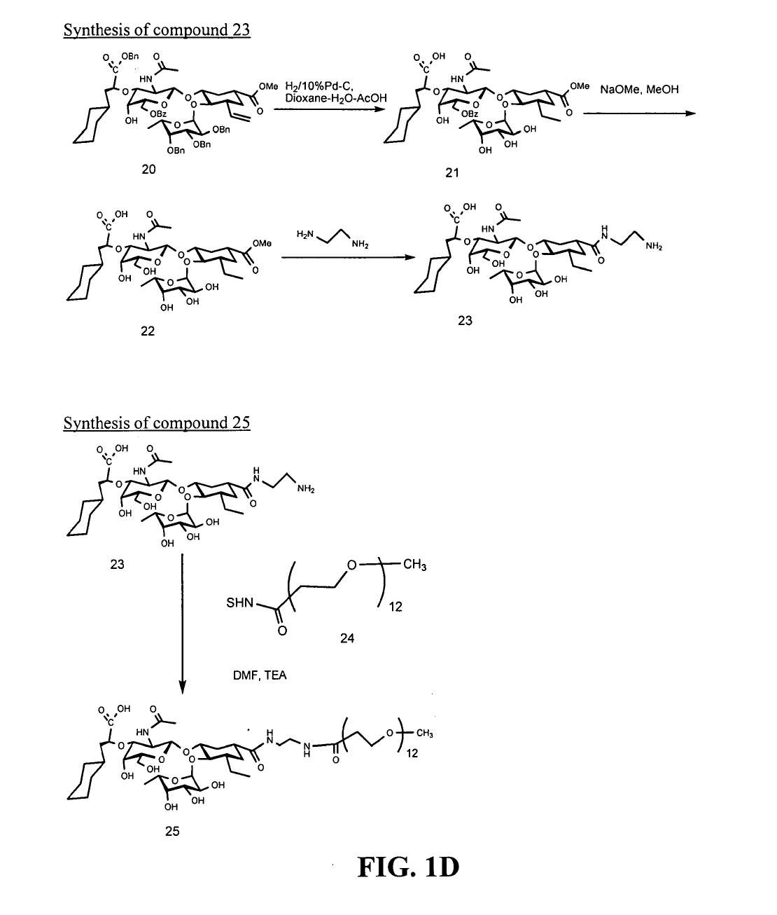

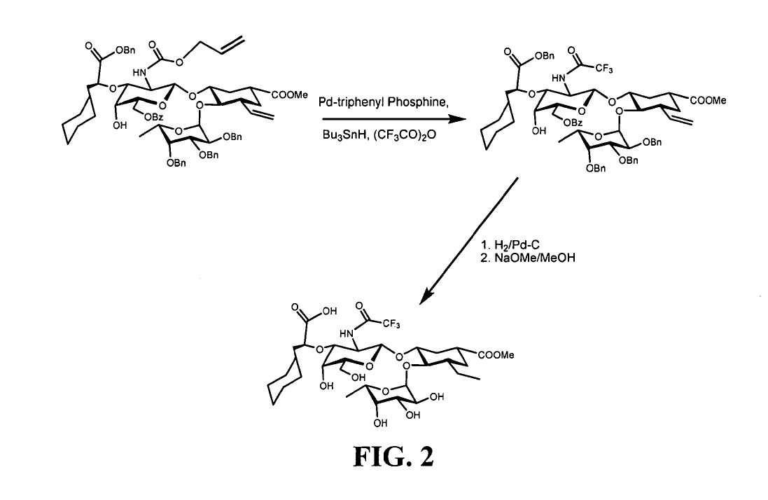

Synthesis of the compounds of Formula (I) may be performed as described herein, including the Examples, using techniques familiar to a person skilled in the art. Synthetic methods for preparing exemplary compounds described herein are described in Example 1. The methods may be used for synthesis of the compounds of Formula (I) by using appropriate reactants for preparation of the specific compound using the techniques and methods described herein, and that are routinely practiced in the art. By way of further example, FIGS. 1 and 2 provide schematics of synthesis schemes for exemplary compounds described herein.

In general, compounds of Formula (I) can be prepared according to the following General Reaction Scheme I:

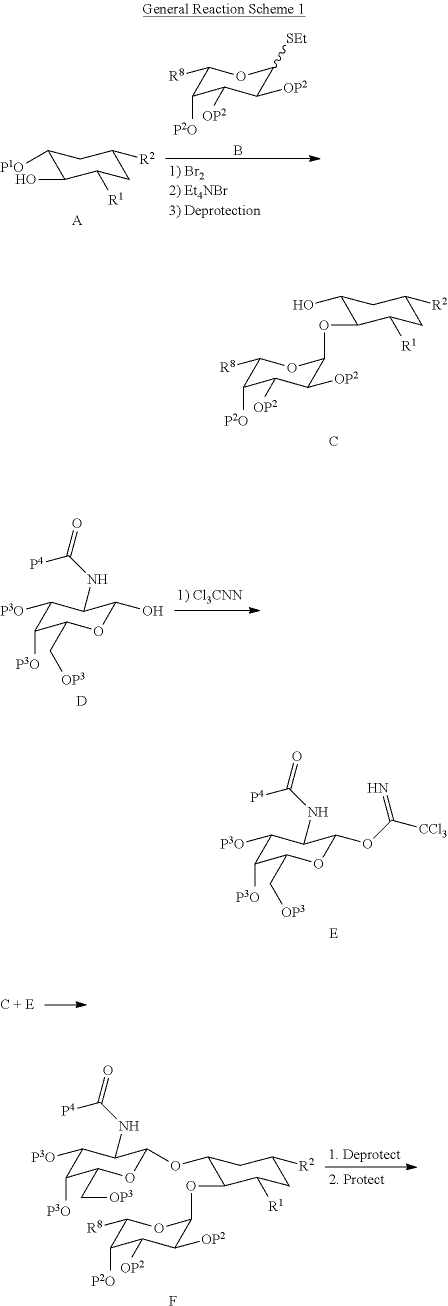

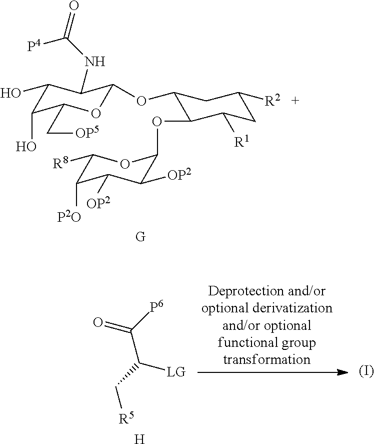

Referring to General Reaction Scheme I, compounds of structure A, wherein R¹ and R² are as defined for Formula (I), or are moieties which can be synthetically converted to R¹ or R², and P¹ is a suitable protecting group, can be purchased from commercial sources or prepared according to methods known in the art. Similarly, compounds of structure B, wherein R⁸ is as defined for Formula (I), or is a moiety which can be synthetically converted to R⁸, and P² is a suitable protecting group, can be purchased from commercial sources or prepared according to methods known in the art. Reaction of A with B, under appropriate conditions (e.g., bromine followed by tetraethylamonium bromide) and subsequent selective removal of P¹ yields compounds of structure C.

In a parallel scheme, compound D, wherein P³ is a suitable protecting group and P⁴ is suitable protecting group or a moiety which can be synthetically manipulated to obtain R³ (as defined for Formula (I)), can be purchased or prepared according to known techniques. Reaction of D with a suitable activating agent (e.g., Cl₃CCN) yields activated compound E. Other suitable means for activating compounds of structure D are known to those of ordinary skill in the art. Coupling of C and E under appropriate conditions yields compounds of structure F.

Selective removal of P³, followed by selective protection yields compounds of structure G, wherein P⁵ is suitable protecting group. Reaction of G with H, wherein P⁶ is suitable protecting group or a moiety which can be synthetically manipulated to obtain R⁴ (as defined for Formula (I)), R⁵ is as defined for Formula (I) and LG is a suitably activated leaving group (e.g., triflate and the like), and deprotection yields exemplary compounds of Formula (I).

It will be appreciated that further synthetic manipulation may be desired to obtain certain compounds of Formula (I). For example, in certain embodiments, P⁴ may be an allyloxy group which can be transformed to obtain an alkyl amide (e.g., methyl). In other examples, R¹ in the above scheme may be an alkenyl moiety, and the synthetic scheme includes reduction of the alkene to an alkyl group. Various other modifications to the above General Reaction Scheme I, such as varying the starting(s) material or modifying any of the reaction products to include other non-hydroxyl moieties at R⁶ and/or R⁷ are possible. Methods for these and other modifications to the above exemplary scheme are well known in the art and described in more detailed in the Examples.

It will also be appreciated by those skilled in the art that in the processes described herein the functional groups of intermediate compounds may need to be protected by suitable protecting groups, even if not specifically described. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

Analogous reactants to those described above may be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

In general, the compounds used in the reactions described herein may be made according to General Reaction Scheme I, Examples 1 and 2, FIGS. 1 and 2 and/or organic synthesis techniques known to those of ordinary skill in this art, starting from commercially available chemicals and/or from compounds described in the chemical literature. “Commercially available chemicals” may be obtained from standard commercial sources including Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hanover, Germany), Spectrum Quality Product, Inc. (New Brunswick, N.J.), TCI America (Portland Oreg.), Trans World Chemicals, Inc. (Rockville Md.), and Wako Chemicals USA, Inc. (Richmond Va.).

Methods known to one of ordinary skill in the art may be identified through various reference books, articles and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

### Methods for Characterizing Glycomimetic Compounds

Biological activity of a glycomimetic compound described herein may be determined, for example, by performing at least one in vitro and/or in vivo study routinely practiced in the art and described herein or in the art. In vitro assays include without limitation binding assays, immunoassays, competitive binding assays and cell based activity assays.

An inhibition assay may be used to screen for antagonists of E-selectin. For example, an assay may be performed to characterize the capability of a compound described herein to inhibit (i.e., reduce, block, decrease, or prevent in a statistically or biologically significant manner) interaction of E-selectin with sLe^{a} or sLe^{x}. The inhibition assay may be a competitive binding assay, which allows the determination of IC₅₀ values. By way of example, E-selectin/Ig chimera may be immobilized onto a matrix (e.g., a multi-well plate, which may be made from a polymer, such as polystyrene; a test tube, and the like); a composition may be added to reduce nonspecific binding (e.g., a composition comprising non-fat dried milk or bovine serum albumin or other blocking buffer routinely used by a person skilled in the art); the immobilized E-selectin may be contacted with the candidate compound in the presence of sLe^{a} comprising a reporter group under conditions and for a time sufficient to permit sLe^{a} to bind to the immobilized E-selectin; the immobilized E-selectin may be washed; and the amount of sLe^{a} bound to immobilized E-selectin may be detected. Variations of such steps can be readily and routinely accomplished by a person of ordinary skill in the art.

Conditions for a particular assay include temperature, buffers (including salts, cations, media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

The source of a compound that is characterized by at least one assay and techniques described herein and in the art may be a biological sample that is obtained from a subject who has been treated with the compound. The cells that may be used in the assay may also be provided in a biological sample. A “biological sample” may include a sample from a subject, and may be a blood sample (from which serum or plasma may be prepared), a biopsy specimen, one or more body fluids (e.g., lung lavage, ascites, mucosal washings, synovial fluid, urine), bone marrow, lymph nodes, tissue explant, organ culture, or any other tissue or cell preparation from the subject or a biological source. A biological sample may further include a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. In some embodiments, the subject or biological source may be a human or non-human animal, a primary cell culture (e.g., immune cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

As described herein, methods for characterizing E-selectin antagonists include animal model studies. Non-limiting examples of animal models for liquid cancers used in the art include multiple myeloma (see, e.g., DeWeerdt, Nature 480:S38-S39 (15 Dec. 2011) doi:10.1038/480S38a; Published online 14 Dec. 2011; Mitsiades et al., Clin. Cancer Res. 2009 15:1210021 (2009)); acute myeloid leukemia (AML) (Zuber et al., Genes Dev. 2009 April 1; 23(7): 877-889). Animal models for acute lymphoblastic leukemia (ALL) have been used by persons of ordinary skill in the art for more than two decades. Numerous exemplary animal models for solid tumor cancers are routinely used and are well known to persons of ordinary skill in the art.

Methods for Treating and/or Preventing Diseases, Disorders, or Conditions

The compounds of the present disclosure and the pharmaceutical compositions comprising at least one of such compounds may be useful in methods for preventing (i.e., reducing the likelihood of occurrence or recurrence of) and/or treating mucositis.

As understood by a person of ordinary skill in the medical art, the terms, “treat” and “treatment,” include medical management of a disease, disorder, or condition of a subject (i.e., patient, individual) (see, e.g., Stedman's Medical Dictionary). In general, an appropriate dose and treatment regimen provide at least one of the compounds of the present disclosure in an amount sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or retard (lessen) an undesired physiological change or disorder, or to prevent or slow or retard (lessen) the expansion or severity of such disorder. As discussed herein, beneficial or desired clinical results from treating a subject include, but are not limited to, abatement, lessening, or alleviation of symptoms that result from or are associated with the disease, condition, or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can include prolonging survival when compared to expected survival if a subject were not receiving treatment. Subjects in need of treatment include those who already have mucositis as well as subjects prone to have or at risk of developing mucositis, and those in which mucositis is to be prevented (i.e., decreasing the likelihood of occurrence of the disease, disorder, or condition).

In some embodiments of the methods described herein, the subject is a human. In some embodiments of the methods described herein, the subject is a non-human animal. A subject in need of treatment as described herein may exhibit at least one symptom or sequelae of mucositis or may be at risk of developing mucositis. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The effectiveness of the compounds of the present disclosure in treating and/or preventing mucositis can readily be determined by a person of ordinary skill in the medical and clinical arts. Determining and adjusting an appropriate dosing regimen (e.g., adjusting the amount of compound per dose and/or number of doses and frequency of dosing) can also readily be performed by a person of ordinary skill in the medical and clinical arts. One or any combination of diagnostic methods, including physical examination, assessment and monitoring of clinical symptoms, and performance of analytical tests and methods described herein, may be used for monitoring the health status of the subject.

In addition, the administration of at least one compound of the present disclosure or pharmaceutical composition comprising at least one such compounds may be in conjunction with one or more other therapies, e.g., for reducing toxicities of therapy. For example, at least one palliative agent to counteract (at least in part) a side effect of a therapy (e.g., anti-cancer therapy) may be administered. Agents (chemical or biological) that promote recovery, or counteract side effects of administration of antibiotics or corticosteroids, are examples of such palliative agents. At least one E-selectin antagonist described herein may be administered before, after, or concurrently with administration of at least one additional anti-cancer agent or at least one palliative agent to reduce a side effect of therapy. When administration is concurrent, the combination may be administered from a single container or two (or more) separate containers.

### Pharmaceutical Compositions and Methods of Using Pharmaceutical Compositions

Also provided herein are pharmaceutical compositions comprising at least one compound of Formula (I). In some embodiments, the pharmaceutical composition further comprises at least one pharmaceutically acceptable ingredient.

In pharmaceutical dosage forms, any one or more of the compounds of the present disclosure may be administered in the form of a pharmaceutically acceptable derivative, such as a salt, and/or it/they may also be used alone and/or in appropriate association, as well as in combination, with other pharmaceutically active compounds.

An effective amount or therapeutically effective amount refers to an amount of a compound of the present disclosure or a composition comprising at least one such compound that, when administered to a subject, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject. In general, the amount of at least one compound of Formula (I) as described herein, that is present in a dose, may range from about 0.01 μg to about 1000 μg per kg weight of the subject. The minimum dose that is sufficient to provide effective therapy may be used in some embodiments. Subjects may generally be monitored for therapeutic effectiveness using assays suitable for the disease or condition being treated or prevented, which assays will be familiar to those having ordinary skill in the art and are described herein. The level of a compound that is administered to a subject may be monitored by determining the level of the compound (or a metabolite of the compound) in a biological fluid, for example, in the blood, blood fraction (e.g., serum), and/or in the urine, and/or other biological sample from the subject. Any method practiced in the art to detect the compound, or metabolite thereof, may be used to measure the level of the compound during the course of a therapeutic regimen.

The dose of a compound described herein may depend upon the subject's condition, that is, stage of the disease, severity of symptoms caused by the disease, general health status, as well as age, gender, and weight, and other factors apparent to a person of ordinary skill in the medical art. Similarly, the dose of the therapeutic for treating a disease or disorder may be determined according to parameters understood by a person of ordinary skill in the medical art.

Pharmaceutical compositions may be administered in any manner appropriate to the disease or disorder to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the pharmaceutical composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical compositions described herein may be administered to a subject in need thereof by any one of several routes that effectively delivers an effective amount of the compound. Non-limiting suitable administrative routes include topical, oral, nasal, intrathecal, enteral, buccal, sublingual, transdermal, rectal, vaginal, intraocular, subconjunctival, sublingual, and parenteral administration, including subcutaneous, intravenous, intramuscular, intrasternal, intracavernous, intrameatal, and intraurethral injection and/or infusion.

The pharmaceutical composition described herein may be sterile aqueous or sterile non-aqueous solutions, suspensions or emulsions, and may additionally comprise at least one pharmaceutically acceptable excipient (i.e., a non-toxic material that does not interfere with the activity of the active ingredient). Such compositions may be in the form of a solid, liquid, or gas (aerosol). Alternatively, the compositions described herein may be formulated as a lyophilizate, or compounds described herein may be encapsulated within liposomes using technology known in the art. The pharmaceutical compositions may further comprise at least one additional component, which may be biologically active or inactive. Non-limiting examples of such components include buffers (e.g., neutral buffered saline or phosphate buffered saline), carbohydrates (e.g., glucose, mannose, sucrose or dextrans), mannitol, proteins, polypeptides, amino acids (e.g., glycine), antioxidants, chelating agents (e.g., EDTA and glutathione), stabilizers, dyes, flavoring agents, suspending agents, and preservatives.

Any suitable excipient or carrier known to those of ordinary skill in the art for use in pharmaceutical compositions may be employed in the compositions described herein. Excipients for therapeutic use are well known, and are described, for example, in Remington. The Science and Practice of Pharmacy (Gennaro, 21^{st} Ed. Mack Pub. Co., Easton, Pa. (2005)). In general, the type of excipient is selected based on the mode of administration, as well as the chemical composition of the active ingredient(s). Pharmaceutical compositions may be formulated for the particular mode of administration. For parenteral administration, pharmaceutical compositions may further comprise water, saline, alcohols, fats, waxes, and buffers. For oral administration, pharmaceutical compositions may further comprise at least one component chosen, for example, from any of the aforementioned excipients, solid excipients and carriers, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose, and magnesium carbonate.

The pharmaceutical compositions (e.g., for oral administration or delivery by injection) may be in the form of a liquid. A liquid pharmaceutical composition may include, for example, at least one the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. In some embodiments, the pharmaceutical composition comprises physiological saline. In some embodiments, the pharmaceutical composition an injectable pharmaceutical composition, and in some embodiments, the injectable pharmaceutical composition is sterile.

For oral formulations, at least one of the compounds of the present disclosure can be used alone or in combination with at least one additive appropriate to make tablets, powders, granules and/or capsules, for example, those chosen from conventional additives, disintegrators, lubricants, diluents, buffering agents, moistening agents, preservatives, coloring agents, and flavoring agents. The pharmaceutical compositions may be formulated to include at least one buffering agent, which may provide for protection of the active ingredient from low pH of the gastric environment and/or an enteric coating. A pharmaceutical composition may be formulated for oral delivery with at least one flavoring agent, e.g., in a liquid, solid or semi-solid formulation and/or with an enteric coating.

Oral formulations may be provided as gelatin capsules, which may contain the active compound or biological along with powdered carriers. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract.

A pharmaceutical composition may be formulated for sustained or slow release. Such compositions may generally be prepared using well known technology and administered by, for example, oral, rectal or subcutaneous implantation, or by implantation at the desired target site. Sustained-release formulations may contain the active therapeutic dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active therapeutic contained within a sustained release formulation depends upon the site of implantation, the rate and expected duration of release, and the nature of the condition to be treated or prevented.

The pharmaceutical compositions described herein can be formulated as suppositories by mixing with a variety of bases such as emulsifying bases or water-soluble bases. The pharmaceutical compositions may be prepared as aerosol formulations to be administered via inhalation. The compositions may be formulated into pressurized acceptable propellants such as dichlorodifluoromethane, propane, nitrogen and the like.

The compounds of the present disclosure and pharmaceutical compositions comprising these compounds may be administered topically (e.g., by transdermal administration). Topical formulations may be in the form of a transdermal patch, ointment, paste, lotion, cream, gel, and the like. Topical formulations may include one or more of a penetrating agent or enhancer (also call permeation enhancer), thickener, diluent, emulsifier, dispersing aid, or binder. Physical penetration enhancers include, for example, electrophoretic techniques such as iontophoresis, use of ultrasound (or “phonophoresis”), and the like. Chemical penetration enhancers are agents administered either prior to, with, or immediately following administration of the therapeutic, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneräs et al., J. Pharm. Pharmacol. 54:499-508 (2002); Karande et al., Pharm. Res. 19:655-60 (2002); Vaddi et al., Int. J. Pharm. 91:1639-51 (2002); Ventura et al., J. Drug Target 9:379-93 (2001); Shokri et al., Int. J. Pharm. 228(1-2):99-107 (2001); Suzuki et al., Biol. Pharm. Bull. 24:698-700 (2001); Alberti et al., J. Control Release 71:319-27 (2001); Goldstein et al., Urology 57:301-5 (2001); Kiijavainen et al., Eur. J. Pharm. Sci. 10:97-102 (2000); and Tenjarla et al., Int. J. Pharm. 192:147-58 (1999).

Kits comprising unit doses of at least one compound of the present disclosure, for example in oral or injectable doses, are provided. Such kits may include a container comprising the unit dose, an informational package insert describing the use and attendant benefits of the therapeutic in treating the pathological condition of interest, and/or optionally an appliance or device for delivery of the at least one compound or composition comprising the same.

### EXAMPLES

### Example 1

### Synthesis of E-Selectin Inhibitor

Exemplary glycomimetic compounds of Formula (I) were synthesized as described in this Example and as shown in the exemplary synthesis schemes set forth in FIGS. 1-2.

Synthesis of Compound 2:

Compound 1 (60 g) was suspended in H₂O (800 mL) and cooled to 0° C. Solid NaHCO₃ (120 g) was added in portion with stirring and then a solution of KI (474.3 g) and I₂ (127 g) in H₂O (800 mL) was added with stirring. Reaction mixture was stirred at room temperature overnight in the dark. Reaction mixture was then extracted with CH₂Cl₂ (3×500 mL). The organic layer was washed with Na₂S₂O₃ solution (2×500 mL) and then the combined aqueous layers were extracted with CH₂Cl₂ (2×300 mL). Organic layers (2100 mL) were combined and washed with cold H₂O (1×500 mL) and cold brine (1×500 mL). The organic layer was dried over Na₂SO₄, filtered, and concentrated to dryness to give compound 2 as light yellow crystals (119 g). Purity: >95% by TLC.

Synthesis of Compound 3:

To a solution of compound 2 (119 g) in THF (1600 mL) was added DBU (119 mL) with stirring at room temperature and the reaction mixture was gently refluxed overnight with stirring. Some precipitate forms and TLC showed no starting material left. The reaction mixture was concentrated to dryness and dissolved in EtOAc (300 mL)mL), washed with 0.5 M HCl (200 mL)mL) until pH 2-3 of the aqueous wash, and then the organic layer was further washed with H₂O (200 mL)mL). Aqueous layers were combined and extracted with EtOAc (3×200 mL)mL) to produce a second organic layer. Combined organic layers (900 mL)mL) were washed with brine, dried (Na₂SO₄), filtered and concentrated to dryness to give compound 3 (58 g). Purity: >95% by TLC.

Synthesis of Compound 4:

To a solution of compound 3 (58 g) in MeOH (800 mL)mL) was added NaHCO₃ (47 g) with stirring. The reaction mixture was stirred under gentle reflux for 3 h, cooled to room temperature, filtered and concentrated to dryness. The residue was dissolved in EtOAc (300 mL)mL) and washed with H₂O. Aqueous layer was extracted with EtOAc (3×100 mL)mL). Combined organic layers (600 mL)mL) were washed with 0.5M HCl (200 mL)mL), H₂O (100 mL)mL), and brine (100 mL)mL), dried (Na₂SO₄), filtered, and concentrated to dryness. The residue was purified by column chromatography (SiO₂, Hexanes-EtOAc 3:1→3:2) to give compound 4 (54 g). Purity: >95% by TLC.

Synthesis of Compound 5:

Compound 4 (31 g) was dissolved in tBuOMe (620 mL)mL) and vinylacetate (166 mL)mL) added with vigorous stirring. Novozyme 435 (1.4 g) was added and vigorous stirring continued for 5.5 h. The reaction mixture was filtered and stored at −20° C. After 12-18 hours, another batch of Novozyme 435 resin (1.4 g) was added and stirred vigorously for 8 h. Resin was filtered and concentrated to dryness. Oily residue was purified by CombiFlash® system (silica) using 0→50% EtOAc/Hexanes to give compound 5 (13.0 g).

Synthesis of Compound 6:

Compound 5 (13.5 g) was dissolved in CH₂Cl₂ (300 mL)mL) under argon and TBDMS-Cl (26.4 g) added with stirring at room temperature under argon. DBU (32.4 mL)mL) was added and stirring continued for overnight at room temperature under argon. MeOH (30 mL)mL) was added and washed with cold saturated solution of NaHCO₃ (200 mL)mL), brine (150 mL)mL). The organic layer was dried (Na2SO4), filtered and concentrated to dryness. The residue was purified by CombiFlash® system (SiO₂) using solvent EtOAc-Hexanes (0-15%) to give compound 6 (18 g). Purity >95% by TLC.

Synthesis of Compound 7:

Compound 6 (12 g) was dissolved in CH₂Cl₂ (400 mL)mL) and cooled to 0° C. m-chloroperbenzoic acid (77%, 19 g) was added and the solution stirred for few hours during which the temperature of the reaction mixture reached to room temperature. The stirring was continued overnight at room temperature. CH₂Cl₂ (300 mL)mL) was added and washed with cold saturated solution of NaHCO₃ (3×400 mL)mL), brine (cold), dried (Na₂SO4), filtered, and concentrated to dryness. The residue was purified by CombiFlash® system (SiO₂) using EtOAc-Hexanes (0→30%) to give 7 (9 g). Purity: >95% by TLC.

Synthesis of Compound 8:

All operation of this step was done in argon atmosphere. CuCN (9.42 g) was dried at 160° C. under vacuum for 40 min, cooled down to room temperature and suspended in THF (80 mL)mL). The mixture was cooled down to −78° C. During this time, tetravinyltin (12 mL)mL) and n-BuLi in hexane (2.5 M, 100 mL)mL) were reacted for 30 min at 0° C. in THF (30 mL)mL). This solution was added to the mixture of CuCN in THF, and the resulting mixture was stirred for 30 min. at −20° C. The mixture was then cooled to −78° C. and to which was added a solution of freshly distilled BF₃.Et₂O (6 mL)mL) in THF (20 mL)mL). The mixture was stirred for 20 min. at −78° C. Compound 7 (5 g) in THF (40 mL)mL) was added and the reaction mixture was stirred at −78° C. for 5 h. MeOH (7 mL)mL) and Et₃N (3 mL)mL) was added and the mixture was concentrated to dryness. The residue was dissolved in EtOAc (200 mL)mL) and washed with saturated solution of NaHCO₃ (2×100 mL)mL), brine (100 mL)mL), dried (Na₂SO₄), filtered, and concentrated to dryness. The residue was purified by CombiFlash® system (SiO₂) using solvent EtOAc-Hexanes (0→5%) to give compound 8 (2.5 g).

Synthesis of Compound 10:

Compound 8 (2.25 g, 7 mmol) was dissolved in toluene (7 mL)mL) and solvent evaporated off. The process was repeated twice and finally dried under vacuum for 15 min. The residue was dissolved in anhydrous CH₂Cl₂ (45 mL)mL) and DMF (45 mL)mL) was added. The solution was stirred under argon at room temperature and molecular sieves (3 g, 4 Å, powdered and flamed dried) added. Et₄NBr (3.3 g, 15.7 mmol, 2.2 equivalents, dried at 200° C. for 2 h) was added and the stirring continued for 1 h at room temperature under argon.

Compound 9 (5.13 g, 10 mmol, 1.42 equivalents) was co-evaporated with toluene (3×20 mL)mL), dried under vacuum, and dissolved in CH₂Cl₂ (45 mL)mL). The reaction mixture was placed in an ice-bath and stirred for 10 min. To this solution was added Br₂ (0.8 ml, 15 mmol, 1.5 equivalents) drop-wise with stirring in the ice-bath. Stirring was continued for 40 min at the same temperature. The ice-bath was removed and cyclohexene (2.1 mL)mL) added slowly with stirring after 10 min. The reaction mixture was stirred for 10 min. and added slowly to the reaction mixture above with stirring at room temperature under argon. Stirring continued for 17 h and then pyridine (4 mL)mL) was added, filtered and the filtrate concentrated to dryness. The residue was dissolved in CH₂Cl₂ (100 mL)mL) and transferred to a separatory funnel. The organic layer was washed with cold brine (2×75 mL)mL), dried (Na₂SO₄), filtered and concentrated to dryness, co-evaporated with toluene (3×50 mL)mL), and dried under vacuum. The residue was dissolved in THF (8 mL) and a solution of TBAF (1 M in THF, 10 ml, 10 mmol, 1.42 equivalents) added with stirring at room temperature. Stirring was continued for 15 h and solvent evaporated off. The residue was dissolved in CH₂Cl₂ (100 mL) and transferred to a separatory funnel, washed with cold brine (2×75 mL), dried (Na₂SO₄), filtered, and concentrated to dryness. The residue was purified by column chromatography (Hexanes-Ethyl acetate from 100% hexanes to 70% hexanes in EtOAc) to give compound 10 (1.6 g, 2.59 mmol, 37% overall in two steps). TLC: 5% EtOAc in hexanes and 33% EtOAc in hexanes.

Synthesis of Compound 12:

Commercially available compound 11 (10 g) was dried overnight under vacuum overnight and added to a solution of NaOMe (5M, 10 mL) in MeOH (200 mL) with stirring at room temperature under argon. Stirring was continued for overnight at room temperature argon, and Et₃N (7 mL) was added followed by allylchloroformate (3.5 mL) dropwise. Stirring was continued for 6 h at room temperature under argon. The reaction mixture was concentrated to dryness and dissolved in pyridine (100 mL). Ac₂O (50 mL) was added at room temperature under argon and stirred at room temperature for overnight. The reaction mixture was concentrated to dryness and purified by column chromatography on CombiFlash® system using EtOAc-Hexanes (0-100%). The desired fractions were collected and concentrated to dryness to give Compound 12 (10.2 g).

Synthesis of Compound 13:

Compound 12 (7.5 g) was dissolved in DMF (140 mL) to which was added NH₄OAC (4.05 g) with stirring. Stirring was continued for overnight at room temperature under argon. The next day the reaction mixture was stirred at 50° C. under argon for 8 h. The reaction mixture was concentrated to dryness and the residue dissolved in EtOAc (150 mL), washed with brine (100 mL), dried (Na₂SO4), filtered, and concentrated to dryness. The residue was purified by column chromatography (SiO₂, Hexanes-EtOAc 2:1→1:2) to give Compound 13 (6 g).

Synthesis of Compound 14:

Compound 13 (6 g) was dissolved in CH₂Cl₂ (50 mL) to which was added CCl₃CN (6 mL) and DBU (0.5 mL). The reaction mixture was stirred at room temperature for 0.5 h, solvent was evaporated off and the residue was purified by column chromatography (silica gel) to give Compound 14 (4.5 g).

Synthesis of Compound 15:

Compound 10 (2 g) and compound 14 (2.1 g) was dissolved in CH₂Cl₂ (40 mL). To this solution were added molecular sieves (4 Å, 0.8 g) and stirred at room temperature for 30 min. The solution was then cooled to 0° C. and BF₃Et₂O (0.25 ml dissolved in 5 mL) is added with stirring at 0° C. The reaction mixture was stirred at 0° C. for 2 h. Et₃N (0.5 mL) was added and the solvent was evaporated off. The residue was purified by column chromatography (silica gel) to give Compound 15 (1.8 g).

Synthesis of Compound 16:

Compound 15 (1.7 g) was treated with 0.01N NaOMe in MeOH (10 mL) for 2 h and neutralized with IR-120 (H⁺) resin, filtered, and concentrated to dryness to give Compound 16 (1.25 g).

Synthesis of Compound 17:

To a solution of compound 16 (1.2 g) in CH₃CN (30 mL) was added Et₃N (0.28 mL) and cooled to 0° C. To this solution was added BzCN (0.35 mg in 10 ml CH₃CN) dropwise during 20 min at 0° C. The reaction mixture was stirred for 1 h at 0° C. and concentrated to dryness. The residue was purified by column chromatography (silica gel) to give compound 17 (0.95 g).

Synthesis of Compound 19:

Compound 17 (0.9 g) was dissolved in MeOH (12 mL). To this solution was added Bu₂SnO (0.4 g) and the mixture was refluxed for 2 h. Solvent was evaporated off and the residual solvent was co-evaporated off with toluene 3 times. The residue was dissolved in dimethoxy ethane (15 mL). To this solution was added CsF (0.8 g) and compound 18 (2.1 g, synthesized as described previously, J. Med. Chem. 42:4909, 1999). The reaction mixture was stirred overnight at room temperature, and the solvent was evaporated off. The residue was purified by column chromatography to give compound 19 (0.8 g).

Synthesis of Compound 20:

Compound 19 (0.7 g) was dissolved in CH₂Cl₂ (20 mL). To this solution was added Pd(Ph)₄ (0.14 g), Bu₃SnH (0.15 mL), and Ac₂O (0.3 mL) and the reaction mixture is stirred at room temperature for 1 h. Solvent was evaporated off and the residue was purified by column chromatography (silica gel) to give compound 20 (0.5 g).

Synthesis of Compound 21:

To a solution of compound 20 (0.45 g) in dioxane-H₂O-AcOH (10:2:1, 2.6 mL) was added 10% Pd-C(0.15 g), and the reaction mixture was shaken at room temperature under positive pressure (20 psi) of hydrogen for 5 h. The solid was filtered off, and the filtrate was concentrated to dryness. The residue was purified by column chromatography (silica gel) to give Compound 21 (0.3 g).

Synthesis of Compound 22:

Compound 21 (0.28 g) was treated with 0.025 N NaOMe in MeOH (5 mL) for 4 h, neutralized with IR-120 (H+) resin, filtered, and the filtrate was concentrated to dryness to give compound 22 (0.21 g).

Synthesis of Compound 23:

Compound 22 (0.18 g) was dissolved in ethylenediamine (2 mL) and stirred at 80° C. for 8 h. Solvent was evaporated off and the residue purified using Sep-pak C18 cartridges to give compound 23 (0.15 g).

Synthesis of Compound 25:

Compound 23 (200 mg) was dissolved into 2 mL DMF. To this solution was added Et₃N (0.1 mL) and then commercially available compound 24 (206 mg). The reaction mixture was stirred at room temperature for 1 h. After evaporation to dryness, the residue was washed with EtOAc (3×4 mL). The solid residue was dissolved in H₂O (2 mL) and the pH of the resulting solution was adjusted to 7.4 by addition of NaOH. The reaction mixture was purified by reverse-phase chromatography (Waters Sep-pak C18 cartridges) using MeOH—H₂O (0-50%) as an eluent. The fractions containing the product were combined, concentrated to dryness and lyophilized to give compound 25 (280 mg). m/z calculated for C₆₀H₁₀₈NaN₃O₂₇=1326.7. Found=1348.7 (M+Na). ¹H-NMR (400 MHz, D₂O): δ 4.94 (d, J=4.0 Hz, 1H), 4.81 (dd, J=6.8 Hz, J=3.2 Hz, 1H), 4.43 (d, J=8.4 Hz, 1H), 3.90 (br t, J=9.2 Hz, 1H), 3.81-3.78 (m, 3H), 3.75-3.71 (m, 2H), 3.70-3.67 (m, 2H), 3.65-3.58 (m, 46H), 3.54-3.52 (m, 2H), 3.48 (br t, J=6.0 Hz, 1H), 3.36 (br d, J=9.6 Hz, 1H), 3.29 (s, 3H), 3.27-3.18 (m, 5H), 2.43 (t, J=6.0 Hz, 2H), 2.25 (bt, J=12.4 Hz, 1H), 2.08-2.05 (m, 1H), 1.97 (s, 3H), 1.79-1.76 (m, 2H), 1.68-1.21 (m, 11H), 1.19-1.04 (m, 8H), 0.86-0.76 (m, 5H). See FIG. 1D.

Synthesis of Compound 45:

Compound 25 (300 mg) was dissolved into 3 mL DMF. Diisopropylethylamine (60 μL) and HATU (131 mg) were added at room temperature. After stirring for 5 minutes, dimethylamine (2.3 mL, 2M solution in THF) was added dropwise. The reaction was stirred at room temperature for 1 hour. The reaction mixture was concentrated to dryness in vacuo. The residue was dissolved in water and loaded onto a 10 g C-18 cartridge. Elution with water followed by 1/I water/MeOH afforded compound 45 (100 mg). m/z calculated for C₆₂H₁₁₄N₄O₂₆=1330.8. Found=1353.6 (M+Na). ¹H NMR 400 MHz (D₂O, set at 4.80 ppm) δ 0.87 (t, J=7.6 Hz, 3H), 0.94-0.99 (m, 2H), 1.20-1.25 (m, 4H), 1.25 (d, J=6.4 Hz, 3H), 1.26-1.45 (m, 4H), 1.52-1.73 (m, 6H), 1.79-1.88 (m, 3H), 2.00 (s, 3H), 2.11-2.19 (br d, 1H), 2.33 (tt, J=12.4 Hz, J=3.2 Hz, 1H), 2.53 (t, J=6.4 Hz, 2H), 2.95 (s, 3H), 3.06 (s, 3H), 3.28 (t, J=12.5 Hz, 1H), 3.31-3.38 (m, 8H), 3.51-3.54 (m, 2H), 3.61 (dd, J=8.0 Hz, J=0.8 Hz, 1H), 3.63 (dd, J=8.0 Hz, J=2.0 Hz, 1H), 3.70 (s, 44H), 3.73-3.76 (m, 1H), 3.78 (t, J=6.0 Hz, 1H), 3.81-3.82 (m, 1H), 3.88 (dd, J=8.0 Hz, J=3.6 Hz, 1H), 3.99 (bs, 1H), 4.54 (dd, 0.1=8.8 Hz, J=2.0 Hz, 2H), 4.91 (q, J=6.8 Hz, 1H), 5.04 (d, J=3.6 Hz, 1H).

Synthesis of Compound 26:

Compound 26 was synthesized as described for compound 25 (see FIG. 1D) except that the PEG reactant had an n of 8 (i.e., 8 repeating PEG units) rather than 12 as for the synthesis of compound 25.

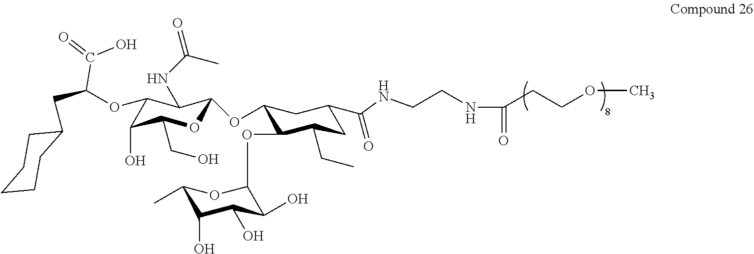

m/z calculated for C₅₂H₉₃N₃O₂₃=1127.6. Found=1151.6 (M+Na). ¹H NMR 600 MHz (D₂O, set at 4.67 ppm) d 0.71 (t, J=7.2 Hz, 3H), 0.76 (br quin, J=12.0 Hz, 2H), 0.99-1.06 (m, 4H), 1.08 (d, J=6.6 Hz, 31-1), 1.15-1.19 (br quin, J=6.6 Hz, 1H), 1.21-1.25 (m, 2H), 1.39-1.48 (m, 5H), 1.50-1.60 (m, 3H), 1.70 (br d, J=10.2 Hz, 2H), 1.91 (s, 3H), 1.99 (m, 1H), 2.16 (br t, J=12.6 Hz, 1H), 2.36 (t, J=6 Hz, 21-1), 3.11-3.15 (m, 2H), 3.18 (t, J=9.6 Hz, 3H), 3.22 (s, 3H), 3.38 (dd, J=7.8 Hz, J=4.2 Hz, 2H), 3.46 (dd, J=4.2 Hz, 1H), 3.47 (s, 1H), 3.52-3.55 (m 27H), 3.56-3.59 (m, 3H), 3.61-3.64 (m, 3H), 3.65 (d, J=3.6 Hz, 1H), 3.72 (dd, J=10.2 Hz, J=3.0 Hz, 1H), 3.80 (d, J=2.4 Hz, 1H), 3.85 (br s, 1H), 3.94 (dd, J=9.6 Hz, J=3.6 Hz, 1H), 4.36 (br s, 1H), 4.77 (q, J=6.6 Hz, 1H), 4.88 (d, I=4.2 Hz, 1H).

Synthesis of Compound 27:

Compound 27 was synthesized as described in FIG. 2.

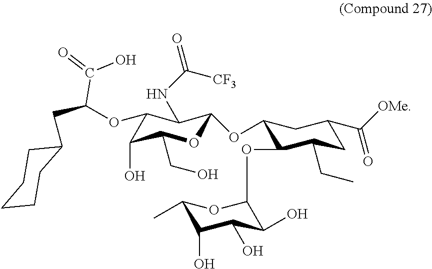

Compound 19 (0.05 g) was dissolved in CH₂Cl₂ (10 mL). To this solution was added Pd[(Ph₃)P]₄ (5 mg), Bu3SnH (0.0011 mL), and (CF3CO)₂O (0.0015 mL) with stirring at room temperature. Stirring was continued for 30 min at room temperature. The reaction mixture was evaporated to dryness under reduced pressure and the residue was purified by column chromatography (silica gel) to give compound 27A (0.030 g).

Compound 27A (0.025 g) was subjected to hydrogenation with 10% Pd-C exactly in same way as described for compound 21 and the solvent was evaporated off after filtering of the catalyst. The residue was treated with NaOMe in MeOH as described for compound 22, neutralized with IR-120 (H+) resin, filtered, and the solvent was evaporated off. The residue was purified by reverse phase (C18) HPLC to give compound 27 (7 mg). m/z calculated for C₃₃H₅₂F₃NO₁₅=759.3. Found=782.3 (M+Na).

Synthesis of Compound 28:

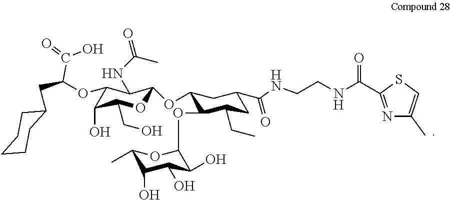

Synthesis Scheme for Compound 28:

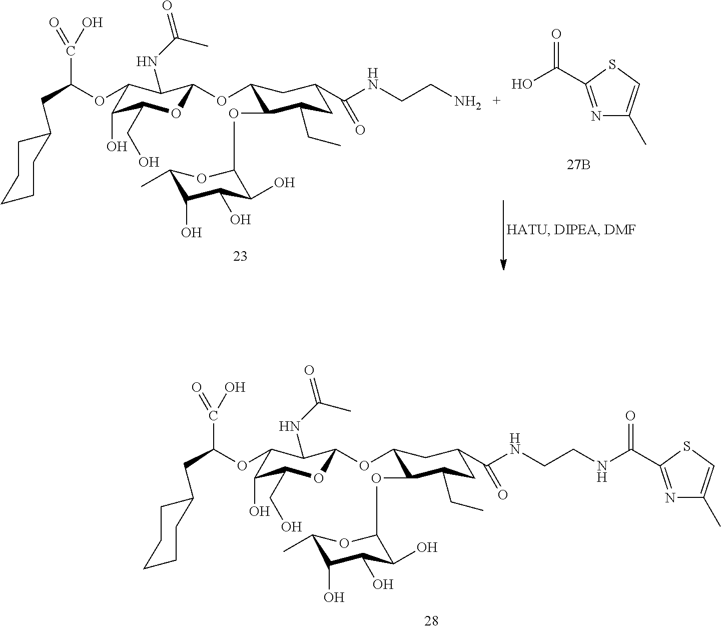

Commercially available compound 27B (0.014 g) was dissolved in DMF (1 mL). To this solution was added DIPEA (0.00175 mL) and HATU (0.038 g) and the reaction mixture was stirred for 2 min at room temperature. Compound 23 (0.035 g) was added and the reaction mixture was stirred for 1 h at room temperature. Solvent was evaporated off and the residue was purified by HPLC (C18) to give compound 28 (17 mg).

Synthesis of Compound 29:

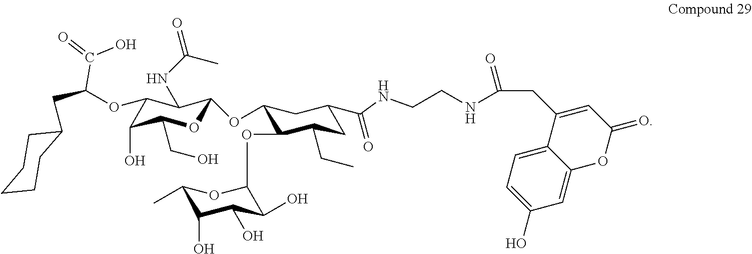

Synthesis Scheme for Compound 29:

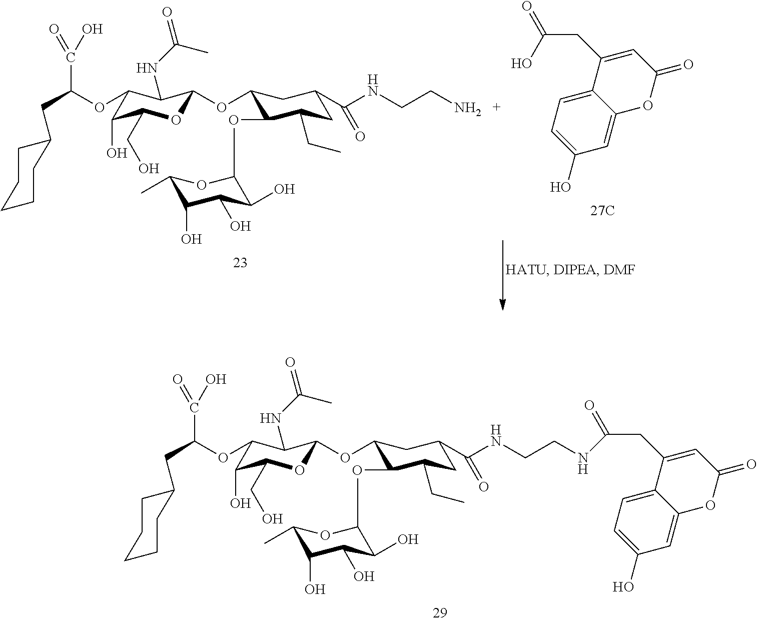

Commercially available compound 27C (0.021 g) was reacted with compound 23 (0.035 g) exactly in the same way as described for compound 28 and purified by HPLC (C18) to give compound 29 (0.020 g).

### Example 2

### E-Selectin Activity—Binding Assay

The inhibition assay to screen for and characterize glycomimetic antagonists of E-selectin is a competitive binding assay, which allows the determination of IC₅₀ values. E-selectin/Ig chimera was immobilized in 96 well microtiter plates by incubation at 37° C. for 2 hours. To reduce nonspecific binding, bovine serum albumin was added to each well and incubated at room temperature for 2 hours. The plate was washed and serial dilutions of the test compounds were added to the wells in the presence of conjugates of biotinylated, sLe^{a} polyacrylamide with streptavidin/horseradish peroxidase and incubated for 2 hours at room temperature.

To determine the amount of sLe^{a} bound to immobilized E-selectin after washing, the peroxidase substrate, 3,3′,5,5′ tetramethylbenzidine (TMB) was added. After 3 minutes, the enzyme reaction was stopped by the addition of H₃PO₄, and the absorbance of light at a wavelength of 450 nm was determined. The concentration of test compound required to inhibit binding by 50% was determined and reported as the IC₅₀ value for each glycomimetic E-selectin antagonist as shown in the table below. IC₅₀ values for exemplary compounds disclosed herein are provided in the following table.

E-Selectin Antagonist Activity
of Glycomimetic Compounds

| Compound | IC₅₀ (μM) |
|---|---|
| 22 | <4.0 |
| 27 | <4.0 |
| 29 | <4.0 |
| 25 | <4.0 |
| 28 | <4.0 |
| 45 | <4.0 |

In addition to reporting the absolute IC₅₀ value as measured above, relative IC₅₀ values (rIC₅₀) are determined by a ratio of the IC₅₀ measured for the test compound to that of an internal control (reference) stated for each assay.

Substitution of the methyl group at the R³ position of compound 22 with a trimethylfluoro (—CF₃) group did not significantly alter the E-selectin antagonist activity of compound 22; however, the substitution did increase the hydrophobicity of the molecule, thereby improving the bioavailability of the glycomimetic compound.

### Example 3

### Mucositis Assay—Intestine Weight

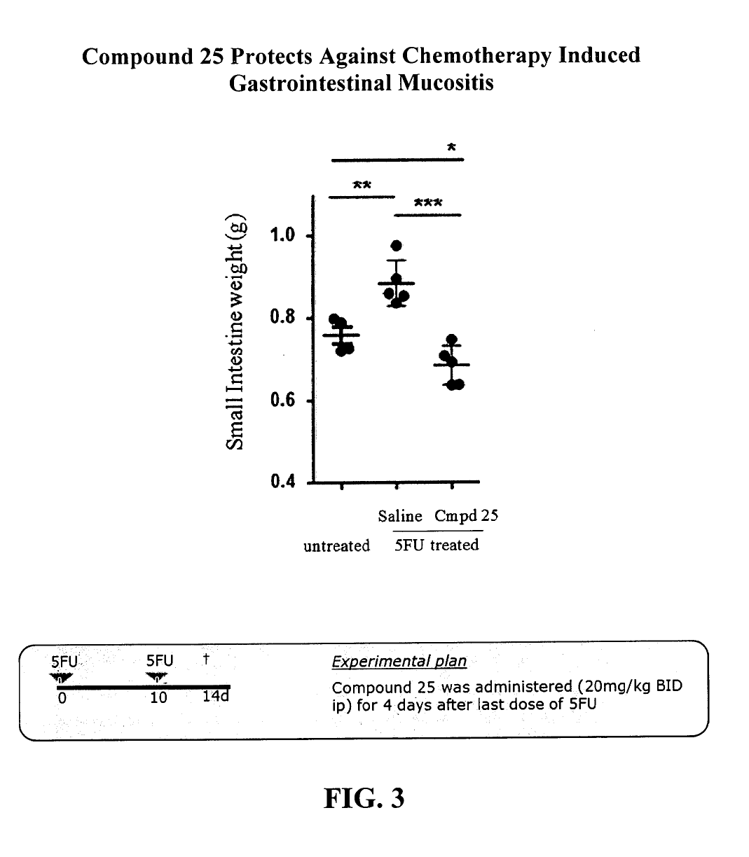

Mice (C57bl/6) were treated with 150 mg/kg of 5-fluorouracil (5-FU) intraperitoneal (ip) on days 0 and 10. After the second injection of 5-FU, the mice were treated with an E-selectin antagonist (20 mg/kg in saline, ip, twice a day) or saline alone (0.15 M NaCl) for 4 days. Mice were then sacrificed and the small intestines were removed and weighed to determine the degree of inflammation. Data showing the results for a representative example is shown in FIG. 3.

### Example 4

### Mucositis Assay—Macrophage Infiltration of the Intestine

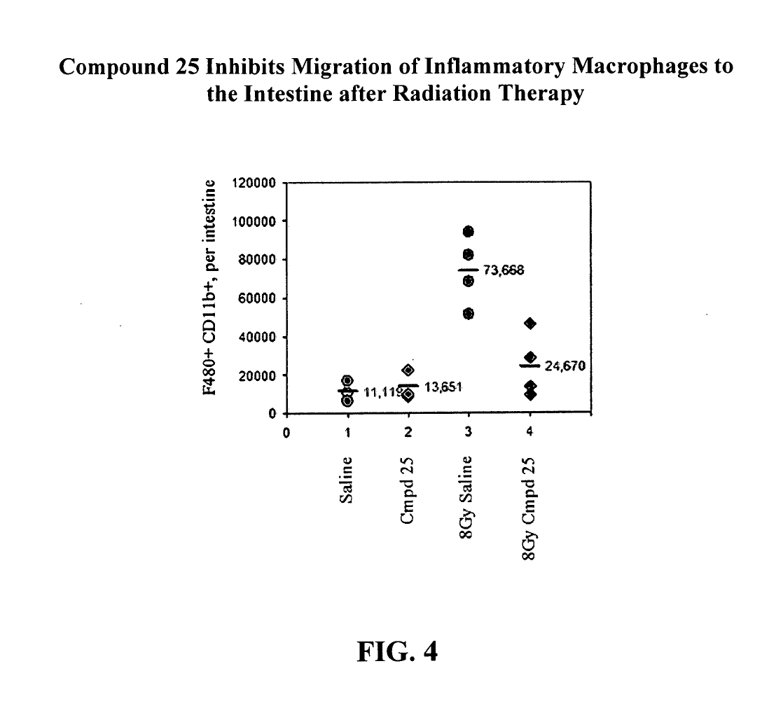

Mice were subjected to whole body irradiation (8.0 Gy) and immediately afterwards treated with an E-selectin antagonist (20 mg/kg in saline, ip, twice a day) or saline alone (0.15 M NaCl) for 6 days. The small intestine was removed at day 6 and digested to release cells. The number of CD11b⁺F4/80⁺ macrophages from the small intestine was determined by flow cytometry. Data showing the results for a representative example is shown in FIG. 4.

What is claimed is:

1. A method for treating and/or preventing mucositis comprising administering to a subject in need thereof an effective amount of at least one compound chosen from E-selectin antagonists, pharmaceutically acceptable salts of E-selectin antagonists, prodrugs of E-selectin antagonists, and pharmaceutically acceptable salts of prodrugs of E-selectin antagonists.

2. The method according to claim 1, wherein the at least one compound is a glycomimetic.

3. The method according to claim 1, wherein the at least one compound is chosen from E-selectin antagonists of Formula (I): 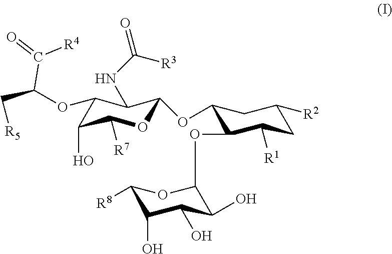
pharmaceutically acceptable salts of E-selectin antagonists of Formula (I), prodrugs of E-selectin antagonists of Formula (I), and pharmaceutically acceptable salts of prodrugs of E-selectin antagonists of Formula (I),
wherein
R¹ is chosen from C₁₋₈ alkyl, C₂₋₈ alkenyl, C₂₋₈ alkynyl, C₁₋₈ haloalkyl, C₂₋₈ haloalkenyl and C₂₋₈ haloalkynyl groups;
R² is chosen from H, -M, and -L-M;
R³ is chosen from C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁴ is chosen from —OH and —NZ¹Z², wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl and C₂₋₈ haloalkynyl groups, wherein Z¹ and Z² may join together to form a ring;
R⁵ is chosen from C₃₋₈ cycloalkyl groups;
R⁶ is chosen from —OH, C₁₋₈ alkyl, C₂₋₈ alkenyl, C₂₋₈ alkynyl, C₁₋₈ haloalkyl, C₂₋₈ haloalkenyl and C₂₋₈ haloalkynyl groups;
R⁷ is chosen from —CH₂OH, C₁₋₈ alkyl, C₂₋₈ alkenyl, C₂₋₈ alkynyl, C₁₋₈ haloalkyl, C₂₋₈ haloalkenyl and C₂₋₈ haloalkynyl groups;
R⁸ is chosen from C₁₋₈ alkyl, C₂₋₈ alkenyl, C₂₋₈ alkynyl, C₁₋₈ haloalkyl, C₂₋₈ haloalkenyl and C₂₋₈ haloalkynyl groups;
L is chosen from linker groups; and
M is a non-glycomimetic moiety chosen from polyethylene glycol, thiazolyl, chromenyl, —C(═O)NH(CH₂)₁₋₄NH₂, C₁₋₈ alkyl, and —C(═O)OY, wherein Y is chosen from C₁₋₄ alkyl, C₂₋₄ alkenyl and C₂₋₄ alkynyl groups.

4. The method according to claim 3, wherein at least one of R¹, R³, R⁶, R⁷ and R⁸ is chosen from C₁₋₈ haloalkyl groups.

5. The method according to claim 4, wherein each C₁₋₈ haloalkyl group is independently chosen from —CH₂X, —CH₂—(CH₂)ₘ—CH₂X, —CHX₂, —CH₂—(CH₂)ₘ—CHX₂, —CX₃ and —CH₂—(CH₂)ₘ—CX₃ groups, wherein each m is independently chosen from integers ranging from 1 to 6 and each X is independently chosen from F, Cl, Br, and I.

6. The method according to claim 5, wherein at least one X is F.

7. The method according to claim 5, wherein at least one C₁₋₈ haloalkyl group is chosen from —CH₂X, —CHX₂, and —CX₃ groups.

8. The method according to claim 7, wherein X is F.

9. The method according to claim 6, wherein R⁴ is N(CH₃)₂.

10. The method according to claim 9, wherein Z is chosen from C₁₋₈ haloalkyl groups.

11. The method according to claim 10, wherein the C₁₋₈ haloalkyl groups are chosen from CH₂X.

12. The method according to claim 11, wherein X is F.

13. The method according to claim 3, wherein R⁵ is cyclohexyl.

14. The method according to claim 3, wherein R² is polyethylene glycol.

15. The method according to claim 1, wherein the at least one compound has the formula: 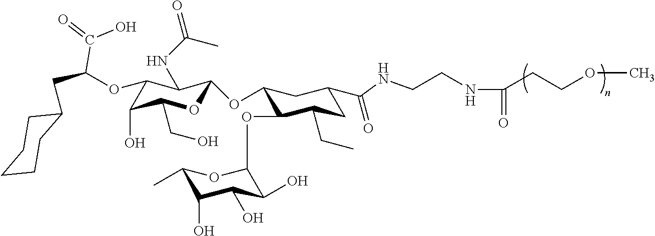
wherein n is chosen from integers ranging from 1 to 100.

16. The method according to claim 1, wherein the at least one compound has the formula:

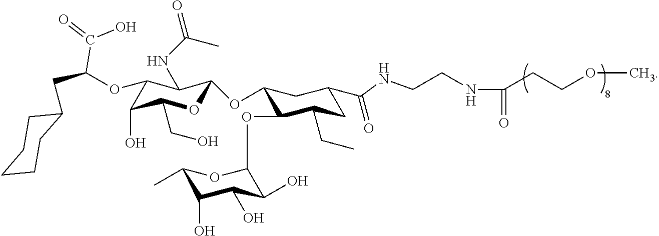

17. The method according to claim 1, wherein the at least one compound has the formula:

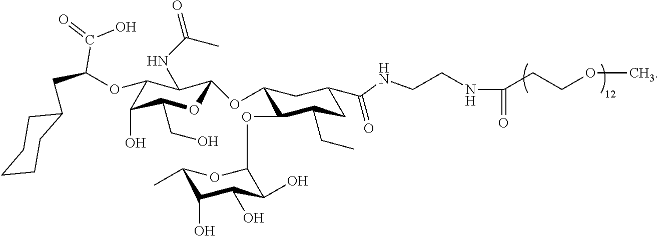

18. The method according to claim 1, wherein the at least one compound has the formula:

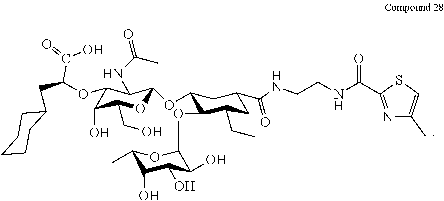

19. The method according to claim 1, wherein the at least one compound has the formula:

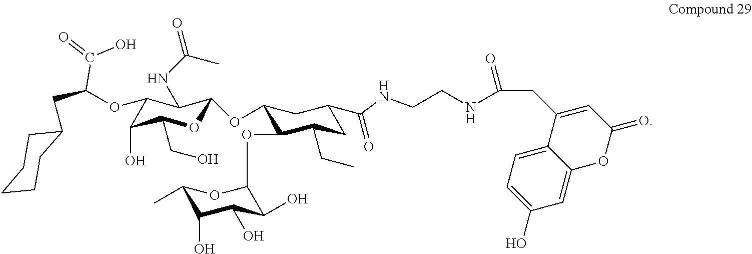

20. The method according to any one of claims 1 to 19, wherein administration of the at least one compound reduces the number of days the patient is afflicted with mucositis.

21. The method according to any one of claims 1 to 19, wherein the mucositis is oral mucositis, esophageal mucositis, and/or gastrointestinal mucositis.

22. The method according to any one of claims 1 to 19, wherein the mucositis is alimentary mucositis.

23. The method according to any one of claims 1 to 19, wherein the mucositis is esophageal mucositis.

24. The method of any one of claims 1 to 19, wherein the mucositis is gastrointestinal mucositis.

25. The method according to any one of claims 1 to 19, wherein the mucositis is oral mucositis.

26. The method according to any one of claims 1 to 19, wherein the subject is afflicted with cancer.

27. The method according to any one of claims 1 to 19, wherein the subject is afflicted with head and neck, breast, lung, ovarian, prostate, lymphatic, leukemic, and/or gastrointestinal cancer.

28. The method according to any one of claims 1 to 19, wherein the mucositis is associated with radiation therapy and/or chemotherapy.

29. The method according to any one of claims 1 to 19, further comprising administering an effective amount of velafermin and/or palifermin.

30. The method according to any one of claims 1 to 19, further comprising administering an effective amount of at least one additional compound chosen from MMP inhibitors, inflammatory cytokine inhibitors, mast cell inhibitors, NSAIDs, NO inhibitors, and antimicrobial compounds.

31. The method according to any one of claims 1 to 19, further comprising administering at least one pharmaceutically acceptable ingredient.
